(19) United States
(12) Patent Application Publication
(10) Pub. No.: US 2023/0416745 A1
(43) Pub. Date: Dec. 28, 2023

(54) HUMAN ORAL MUCOSA STEM CELL SECRETOME

(71) Applicant: RAMOT AT TEL-AVIV UNIVERSITY LTD., Tel Aviv (IL)

(72) Inventor: Sandu PITARU, Tel Aviv (IL)

(21) Appl. No.: 18/366,403

(22) Filed: Aug. 7, 2023

Related U.S. Application Data
(62) Division of application No. 16/630,954, filed on Jan. 14, 2020, now abandoned.
(60) Provisional application No. 62/533,056, filed on Jul. 16, 2017.

(51) Int. Cl. C12N 15/113; A61P 17/02

(52) U.S. Cl. CPC C12N 15/113; A61P 17/02; C12Y 115/01001; A61K 31/713

(57) ABSTRACT

The present invention provides secretome derived from human oral mucosa stem cells (hOMSC), and cell-free compositions comprising hOMSC-derived secretome. Methods for obtaining, manipulating and using hOMSC-derived secretome in therapy, cosmetics and tissue regeneration are also provided.

### FIELD OF THE INVENTION

The present invention is in the fields of stem cells and regenerative medicine. In particular, the present invention provides compositions of secretome derived from human oral mucosa stem cells. Methods for obtaining, manipulating and using stem cell secretome in therapy are also provided.

### BACKGROUND OF THE INVENTION

Human oral mucosa-derived stem cells (hOMSC) are a unique stem cell population derived from the lamina propria of the oral mucosa (Marynka-Kalmani et al. 2010). hOMSC express a unique immunophenotype that consists of markers of embryonic stem cells, neural crest stem cells and mesenchymal stem cells. Global gene analysis identified that the transition of hOMSC from in vivo to in vitro resulted in the differential expressions of genes that are involved in the development of the neural crest cell lineages during the embryonic and fetal developmental stages of the mammalian organism.

The neural crest is a temporary developmental structure that gives rise to a variety of cell lineages of ectodermal and mesenchymal origin including neuronal and glial lineages and chondroblastic, osteoblastic, adipocytic and fibroblastic lineages, respectively. hOMSC have also been shown in vitro and in vivo to differentiate into these cells lineages (Marynka-Kalmani et al. 2010, Treves-Manushevitz et al. 2013, Ganz et al. 2014a, 2014b).

hOMSC that were differentiated into dopaminergic-like neurons or astrocyte-like cells and transplanted in vivo were found to have therapeutic effects in animal models (Ganz et al. 2014a, 2014b). Moreover, even naïve hOMSC were shown to have some therapeutic activity be ineffective in these animal models, their therapeutic effect being similar to that of the placebo.

Recently, it has been shown that embryonic and adult stem cells are also a reach source of Extracellular vesicles (Desrochers et al. 2016, Konala et al 2016). Extracellular Vesicles (EV) are vesicles released from the cytoplasm of eukaryotic cells ranging in size from 50 nm to 1.5-2 microns.

EV are majorly divided into 2 main categories according to their biogenesis and size: microvesicles or shed microvesicles having a size range of 50-1500 nm; and exosomes having a size range of 30-120 nm. Exosomes are lipid bilayer membrane vesicles derived from the luminal membrane of multi-vesicular bodies, which are constitutively released by fusion with the cell membrane. The biogenesis of microvesicles and exosomes is different. Microvesicles are formed at the plasma membrane by budding and fission from the membrane. Exosomes are derived from the endosomal and Golgi systems and rooted to the cell surface at least in part by the endosomal sorting complex required for transport where they undergo exocytosis.

Extracellular vesicles contain cargo that is composed of proteins, lipids, nucleic acids (Desrochers et al. 2016, Xu et al. 2016). The EV content is heterogeneous and in a dynamic state, depending on the cell's origin, its physiological and pathological state, and on the cellular release site. The composition of exosomes can be different from the cells of their origin due to the selective sorting of cargo into exosomes. EV's cargo has been shown to serve as a method for cell-cell communication in addition to the classical ways of cell-cell contact and the secretion of soluble factors for paracrine and autocrine effect. Most of the knowledge on EV biological effect originates from work done on cancer cells. It has been shown vitro and in vivo that the EV's cargo induces cancer cell proliferation and survival and angiogenesis as well as tumor fibroblast migration, survival and growth (Antonyak et al. 2015). Exosomes have been proven to be carriers of genetic materials and been nominated as biomarkers for cancer diagnosis and prognosis and proposed for monitoring of therapeutic efficacy. Exosome-based delivery of tumor vaccines and drugs is currently evaluated as therapeutic strategy for cancer (Gue et al., 2017).

Conditioned medium from adult stem cells derived from bone marrow and adipose tissue was found to have therapeutic potential in cardiac ischemia and in wound healing (Lai et al. 2010, Hu et al. 2016).

There is accumulating evidence that the content of the cargo differs between the microvesicles and the exosomes and that difference is controlled by the origin of the cells (Kanada et al. 2015). Furthermore, recent data indicate that the nature of the cargo is cell-specific both for EV released by various types of cancer cells and for those released by different types of stem cells (Villarroya-Beltri et al. 2014, Lopez-Verrilli MA et al. 2016).

WO 2008/132722 discloses the lamina propria of the mucosa of the gastrointestinal tract and in particular of the oral mucosa, as a source for pluripotent adult stem cells.

WO 2013076726 discloses stem cells derived from the lamina propria of the oral mucosa (OMSC), as a source for selective differentiation into different neural lineages and their use in induction or preservation of neurogenesis, and for therapy of neurodegenerative and psychiatric disorders and in loss of neural tissue due to trauma.

US2016/0256496 relates to gingival fibroblast-derived product, e.g. conditioned medium, and its use in methods for the prevention or treatment of orthopedic pathologies such as osteoarthritis and rheumatoid arthritis.

WO/2017/001649, WO/2016/082882 and WO/2016/083500 of Med Cell Ltd., disclose specific methods of producing a secretome secreted by mesenchymal stem cells or dendritic cells.

WO/2014/057097 discloses a method for modulating the secretome of adult human mesenchymal stem cells by co-culturing adult human mesenchymal stem cells and adult fully differentiated cardiomyocytes in an appropriate culture medium to obtain preconditioned adult human mesenchymal stem cells.

There remains an unmet need for compositions useful in prevention and treatment of diseases and disorders and in promoting tissue regeneration. Such compositions may advantageously be derived from unique, expandable and readily accessible source.

### SUMMARY OF THE INVENTION

Conditioned media, or secretomes of human stem cells derived from the lamina propria of the oral mucosa (hOMSC), are now disclosed as therapeutic compositions. The invention is based in part on the finding that the composition of naïve hOMSC secretome is unique, and therefore has unique therapeutic potential, which is advantageous over the secretomes of naïve stem cells derived from other sources. Furthermore, hOMSC stimulation may result in a cell response that is reflected in the change in the secretome content and therefore in its therapeutic capacity. It is also envisaged that the secretome resulting from said hOMSC stimulation is unique to hOMSC-stimulated cells and that the same stimulation will result in a different secretome if applied to adult stem cells derived from other sources.

It is herein disclosed for the first time that the secretome of naïve hOMSC has a unique signature which is different than secretomes of other stem cells in existence, absence or relative quantity of cytokines, chemokines and nucleic acids.

It is also disclosed herein that hOMSC and cell-free compositions comprising secretomes derived thereof are capable of enhancing diabetic wound healing, suggesting potential use of hOMSC secretomes in promoting new vasculature, cell proliferation and connective tissue formation. Unexpectedly, hOMSC and their secretome activity in wound healing is superior to stem cells and secretomes derived from other sources.

Secretome according to the present invention derived from the accessible, reproducible and expendable source of naïve hOMSC cells, is simple to obtain and use without the need of induction of differentiation of the cells.

The present invention provides, according to one aspect, a cell-free composition, comprising substances secreted from human oral mucosa stem cells (hOMSC-derived secretome), together with at least one carrier, excipient or diluent.

The cell-free compositions comprising hOMSC-derived secretomes according to the present invention are unique in their content and are different from secretomes of stem cells of other sources.

According to some embodiments, the hOMSC are naïve.

According to some embodiments, the cell-free composition comprises:
(i) at least one protein from the group consisting of: Stromal cell-derived factor 1 (CXCL12/SDF1), Superoxide dismutase [Cu—Zn] (SOD1), Mesencephalic astrocyte-derived neurotrophic factor (MANF), Cystatin-C(CST3), Galectin-1 (LGALS1), Glia-derived nexin (SERPINE2), Insulin-like growth factor II (IGF2), Latent-transforming growth factor beta-binding protein 1 (LTBP1), Latent-transforming growth factor beta-binding protein 2 (LTBP2), Latent-transforming growth factor beta-binding protein 3 Fragment (LTBP3), Latent-transforming growth factor beta-binding protein 4 (LTBP4), Neuroblast differentiation-associated protein (AHNAK) and Pigment epithelium-derived factor (SERPINF1/PEDF); or
(ii) at least one protein selected from the group consisting of: hepatocyte growth factor (HGF), placental growth factor (PIGF), macrophage colony-stimulating factor (MCSF), vascular endothelial growth factor (VEGF), granulocyte colony stimulating factor (GCSF), Macrophage Inflammatory Protein-3 (MIP-3a), growth-regulated oncogene-alpha (GRO-a or CXCL1), Macrophage-Derived/CCL22 Chemokine (MDC or CCL22), growth-regulated oncogene (GRO), IGFBP-2, neurotrophin-4 (NT-4), monocyte chemoattractant protein 2 (MCP-2/CCL8), insulin growth factor-1 (IGF-1), Granulocyte-macrophage colony-stimulating factor (GM-CSF), Interleukin-2 (IL-2) and Brain-Derived neurotrophic factor (BDNF); or
(iii) at least one protein selected from the group consisting of: 1 SV, 3 SV, ACTG2, ADAM10, ADAMTSL1, ADM, ANXA4, APOD, CALM2, CD109, CD59, CDH6, CFD, COL15A1, COL1A2, COLEC12, CTHRC1, CTSC, CTSL, CXCL12, DCD, DDAH2, DKK1, DSG1, DSP, DSTN, ECH1, EDIL3, EFEMP1, ELN, FLG, GNB2, GREM2, H3F3B, HBA1, HIST1H2AH, HIST1H2BK, HIST1H4A, HMGN2, HNRNPAB, HSP90AA1, HSPA1A, HSPG2, IGFBP5, JUP, KHSRP, LDHA, MNB2, LTBP4, MAN1A1, MFAP4, MMP1, MMP14, MT2A, NBL1, OMD, PFN1, PI16, PSG5, PSMB6, PTGDS, RARRES2, SLIT3, SPOCKI, SPTBN4, STOM, TMSB10, TMSB4X, TNFAIP6, TNXB, TPI1, TUBA1C, UBC, VIT and WNT5A; or
(iv) at least one microRNA (miRNA) selected from the group consisting of: hsa-miR-4454+hsa-miR-7975, hsa-miR-23a-3p, hsa-let-7b-5p, hsa-miR-612, hsa-miR-125b-5p, hsa-miR-3144-3p, hsa-miR-199a-3p+hsa-miR-199b-3p, hsa-miR-191-5p, hsa-miR-100-5p, hsa-miR-127-3p, hsa-miR-1260a, hsa-miR-378h, hsa-miR-379-5p, hsa-miR-376a-3p, hsa-let-7i-5p, hsa-miR-526a+hsa-miR-518c-5p+hsa-miR-518d-5p, hsa-miR-212-3p, hsa-miR-520c-3p, hsa-miR-28-5p, hsa-miR-758-3p+hsa-miR-411-3p, hsa-miR-29a-3p, hsa-miR-1206, hsa-miR-1286, hsa-miR-514a-3p, hsa-miR-548ah-5p, hsa-miR-184, hsa-miR-543, hsa-miR-626, hsa-miR-339-3p, hsa-miR-1234-3p, hsa-miR-155-5p, hsa-miR-888-5p, hsa-miR-542-3p, hsa-miR-514b-5p, hsa-miR-548m, hsa-miR-30e-5p and hsa-miR-1290;
or combinations thereof.

According to some embodiments, the cell-free composition comprises plurality of substances from (i), (ii), (iii), or (iv).

According to yet other embodiments, the cell-free composition comprises at least one protein from (i), at least one protein from (ii), at least one protein of (iii), and optionally at least one miRNA from (iv).

According to some specific embodiments, the cell-free composition of hOMSC-derived secretomes comprises at least one factor selected from the group consisting of: Stromal cell-derived factor 1 (CXCL12/SDF1), Mesencephalic astrocyte-derived neurotrophic factor (MANF), Superoxide dismutase [Cu—Zn] (SOD1), hepatocyte growth factor (HGF), placental growth factor (PIGF), macrophage colony-stimulating factor (MCSF) and vascular endothelial growth factor (VEGF).

According to other embodiments the cell-free composition of hOMSC-derived secretomes comprises at least one microRNA molecule selected from the group consisting of: hsa-miR-4454+hsa-miR-7975, hsa-miR-23a-3p, hsa-let-7b-5p, hsa-miR-612, hsa-miR-125b-5p, hsa-miR-3144-3p, hsa-miR-199a-3p+hsa-miR-199b-3p, hsa-miR-191-5p, hsa-miR-100-5p, hsa-miR-127-3p, hsa-miR-1260a, hsa-miR-378h, hsa-miR-379-5p, hsa-miR-376a-3p, hsa-let-7i-5p, hsa-miR-526a+hsa-miR-518c-5p+hsa-miR-518d-5p, hsa-miR-212-3p, hsa-miR-520c-3p, hsa-miR-28-5p, hsa-miR-758-3p+hsa-miR-411-3p, hsa-miR-29a-3p, hsa-miR-1206, hsa-miR-1286, hsa-miR-514a-3p, hsa-miR-548ah-5p, hsa-miR-184, hsa-miR-543, hsa-miR-626, hsa-miR-339-3p, hsa-miR-1234-3p, hsa-miR-155-5p, hsa-miR-888-5p, hsa-miR-542-3p, hsa-miR-514b-5p, hsa-miR-548m, hsa-miR-30e-5p and hsa-miR-1290.

According to some specific embodiments, the cell-free composition of hOMSC-derived secretomes comprises at least six microRNA molecules selected from the group consisting of: hsa-miR-4454+hsa-miR-7975, hsa-miR-23a-3p, hsa-let-7b-5p, hsa-miR-612, hsa-miR-125b-5p, hsa-miR-3144-3p, hsa-miR-199a-3p+hsa-miR-199b-3p, hsa-miR-191-5p, hsa-miR-100-5p, hsa-miR-127-3p, hsa-miR-1260a, hsa-miR-378h, hsa-miR-379-5p, hsa-miR-376a-3p, hsa-let-7i-5p, hsa-miR-526a+hsa-miR-518c-5p+hsa-miR-518d-5p, hsa-miR-212-3p, hsa-miR-520c-3p, hsa-miR-28-5p, hsa-miR-758-3p+hsa-miR-411-3p, hsa-miR-29a-3p, hsa-miR-1206, hsa-miR-1286, hsa-miR-514a-3p, hsa-miR-548ah-5p, hsa-miR-184, hsa-miR-543, hsa-miR-626, hsa-miR-339-3p, hsa-miR-1234-3p, hsa-miR-155-5p, hsa-miR-888-5p, hsa-miR-542-3p, hsa-miR-514b-5p, hsa-miR-548m, hsa-miR-30e-5p and hsa-miR-1290.

According to some embodiments, the cell-free compositions comprises at least one protein in a significant higher concentration than the concentration of said protein in secretome derived from other sources of stem cells. According to some specific embodiments, the cell-free composition comprises at least one protein of (i), (ii) or (iii) in a significant higher concentration than in secretome derived from other sources of stem cells.

According to some embodiments, the at least one protein present in the cell-free composition of hOMSC-derived secretome, in a significant higher concentration than in secretome derived from other sources of stem cells, is selected from the group consisting of: Stromal cell-derived factor 1 (CXCL12/SDF1, P48061), Superoxide dismutase [Cu—Zn](SOD1, P00441), Mesencephalic astrocyte-derived neurotrophic factor (MANF, P55145), hepatocyte growth factor (HGF), placental growth factor (PIGF), macrophage colony-stimulating factor (MCSF), and vascular endothelial growth factor (VEGF).

According to certain embodiments, the secretome comprises at least one protein selected from the group consisting of: Stromal cell-derived factor 1 (CXCL12/SDF1), Mesencephalic astrocyte-derived neurotrophic factor (MANF), Superoxide dismutase [Cu—Zn] (SOD1), hepatocyte growth factor (HGF), placental growth factor (PIGF), macrophage colony-stimulating factor (MCSF), vascular endothelial growth factor (VEGF), growth-regulated oncogene (GRO), granulocyte colony stimulating factor (GCSF), Macrophage Inflammatory Protein-3 (MIP-3a), growth-regulated oncogene-alpha (GRO-a or CXCL1), Macrophage-Derived/CCL22 Chemokine (MDC or CCL22), insulin like growth factor binding protein 2 (IGFBP-2), neurotrophin-4 (NT-4), monocyte chemoattractant protein 2 (MCP-2), also known as Chemokine (C—C motif) ligand 8 (CCL8), insulin growth factor-1 (IGF-1), insulin like growth factor-2, Cystatin-C(CST3), Galectin-1 (LGALS1), Glia-derived nexin (SERPINE2), Latent-transforming growth factor beta-binding protein 1 (LTBP1), Latent-transforming growth factor beta-binding protein 2 (LTBP2), Latent-transforming growth factor beta-binding protein 3 (LTBP3), Latent-transforming growth factor beta-binding protein 4 (LTBP4), Neuroblast differentiation-associated protein (AHNAK), Pigment epithelium-derived factor (SERPINF1), and Granulocyte-macrophage colony-stimulating factor (GM-CSF), in a significant higher concentration than the concentration of said protein in secretome derived from skin or bone marrow stem cells.

According to certain embodiments, the secretome comprises at least one protein selected from the group consisting of: hepatocyte growth factor (HGF), placental growth factor (PIGF), macrophage colony-stimulating factor (MCSF), and vascular endothelial growth factor (VEGF), in a significant higher concentration than the concentration of said protein in secretome derived from skin or bone marrow stem cells.

According to other embodiments, the hOMSC-derived secretome comprises at least one protein in a significant lower concentration than the concentration of said protein in secretome derived from other sources of stem cells.

According to some embodiments, the cell-free composition of hOMSC-derived secretome comprises at least one protein in a significant lower concentration than in secretome derived from other sources of stem cells.

According to certain embodiments, the at least one protein present in a significant lower concentration than in secretome derived from other sources of stem cells, is selected from the group consisting of: monocyte chemotactic protein-3 (MCP-3/CCL7), Epithelial-neutrophil activating peptide or C—X—C motif chemokine 5 (ENA-78 or CXCL5), leptin, Fms-related tyrosine kinase 3 ligand (Flt-3 ligand), interleukin-6 (IL-6), Monokine induced by gamma interferon (MIG or CXCL9), and interleukin 8 (IL-8), in a significant lower concentration than the concentration of said protein in secretome derived from skin or bone marrow stem cells. Each possibility represents a separate embodiment of the present invention.

According to some embodiments, the protein and nucleic acid content of the hOMSC-derived secretome is different than the protein and nucleic acid content of secretome of any other stem cells.

According to certain embodiments, the secretome comprises at least one protein involved in the homeostasis of the nervous system.

According to some embodiments, the at least one protein involved in the homeostasis of the nervous system may be selected from: Cystatin-C, Galectin-1, Glia-derived nexin, Insulin-like growth factor II, (IGF2), Latent-transforming growth factor beta-binding protein 1 (LTBP1), Latent-transforming growth factor beta-binding protein 2 (LTBP2), Latent-transforming growth factor beta-binding protein 3 (LTBP3), Latent-transforming growth factor beta-binding protein 4 (LTBP4), Mesencephalic astrocyte-derived neurotrophic factor (MANF), Neuroblast differentiation-associated protein (AHANK), Pigment epithelium-derived factor (PEDF), Stromal cell-derived factor 1 (SDF1), and Superoxide dismutase [Cu—Zn] (SODC). Each possibility represents a separate embodiment of the present invention.

hOMSC-derived secretome according to the present invention comprises substances secreted or released into the medium in which they are grown or maintained. Such a medium is herein termed conditioned medium.

According to some embodiments the hOMSC-derived secretome and the cell-free compositions comprises extracellular vesicles (EV).

According to some embodiments, the hOMSC-derived secretome and the cell-free compositions comprises microvesicles.

According to other embodiments, the hOMSC-derived secretome and the cell-free compositions comprises exosomes.

According to some embodiments, the hOMSC-derived secretome and the cell-free compositions comprises microvesicles and exosomes.

According to some embodiments, the composition hOMSC-derived secretome and the cell-free compositions comprises soluble factors.

According to some embodiments, the soluble factors are selected from the group consisting of: proteins, peptides, hormones, DNA and RNA species, oligo and polynucleotides, and combinations thereof.

According to some embodiments, the soluble factors are molecules having a molecular size of 1,000 Daltons (Da) or higher.

According to some specific embodiments, the soluble factors are molecules having a molecular size between, for example 1,000-10,000 Da; 1,000-3000 Da; 1,000-5,000 Da; 2,000-6,000 Da; 5,000-10,000 Da; 7,000-10,000 Da, 10,000-30,000 Da; 10,000-50,000 Da etc., or even higher molecular size. Each possibility represents a separate embodiment of the present invention.

According to some embodiments the conditioned medium of the hOMSC is concentrated using methods known in the art, to yield hOMSC secretome comprising constituents in concentration higher than that of the conditioned medium.

According to some embodiments, the secretome is derived from hOMSC which were subjected to stimulation or condition which influenced the content of the secretome.

According to some embodiments, the stimulation or condition may include, but is not limited to: chemical, physical, substrate and/or biological stimulation.

According to some embodiments, the cell-free composition is a pharmaceutical composition comprising a hOMSC-derived secretome together with a pharmaceutically acceptable carrier, excipient or diluent.

According to some embodiments, the cell-free composition is a cosmetic composition comprising a hOMSC-derived secretome together with an acceptable carrier, excipient or diluent suitable for cosmetic application.

According to some embodiments, the cell-free composition comprising hOMSC-derived secretome, is for use in tissue remodeling or tissue regeneration.

According to some embodiments, a pharmaceutical composition according to the present invention is provided for use in enhancing wound healing, preventing or reducing scar formation, enhancing scar healing, or enhancing cartilage- or bone-formation.

According to some embodiments, the cell-free composition comprising hOMSC-derived secretome, is for use in promoting or accelerating diabetic wound healing.

The present invention provides, according to another aspect, a method of producing a cell-free secretome from hOMSC, and wherein the method comprises the steps of:
i. isolating hOMSC by explantation or enzymatic digestion;
ii. expanding hOMSC in a culture medium;
iii. replacing the culture medium with a basal medium;
iv. culturing hOMSC in the basal medium for a period ranging from 1 hour to 120 hours;
v. harvesting the medium from the cultures; and
vi. optionally concentrating the medium by 1.1-10,000 folds.

According to some embodiments, the isolated hOMSC are naïve cells.

According to some embodiments, during or following step (iv), the hOMSC are subjected to stimulation or condition which influence the content of the secretome. According to some embodiments, said stimulation is selected from the group consisting of chemical, physical, substrate or biological stimulation.

Naïve or stimulated hOMSC used according to the present invention for production of secretomes and cell-free compositions are maintained and expanded in tissue culture in an undifferentiated state.

Pharmaceutical and cosmetic cell-free compositions comprising hOMSC-derived secretome, may be used according to the present invention, for the repair and regeneration of organs and tissues that were totally or partially destroyed by mechanical trauma, chemical injuries, radiation, and heat or any other type of iatrogenic injuries. Examples of such injuries include but are not limited to: contusion of the central nervous system, spinal injuries, section of the spinal cord, peripheral nerve crush or section, burns, neuropathy and cardiopathy due to chemotherapy, bone fractures, tendon and ligament rupture. Each possibility represents a separate embodiment of the present invention.

According to some embodiments the compositions of the present invention comprises secretome derived from autogenous hOMSC, namely, the treated individual acts as a donor for the hOMSC for producing the secretome.

According to other embodiments the compositions of the present invention comprises secretome derived from allogeneic hOMSC, namely, a donor unrelated to the patient acts as a donor for the hOMSC for producing the secretome.

According to yet additional aspect the present invention provides a method of preventing or treatment of a disease or disorder comprising administering to a subject in need thereof a cell-free composition comprising hOMSC-derived secretome.

Any disease or disorder eligible for prevention or treatment with stem cells may be treated or prevented with a composition according to the invention.

According to some embodiment, a disease or disorder eligible for prevention or treatment with the compositions of the invention is selected from the group consisting of:
i. inflammatory diseases (e.g. osteoarthritis);
ii. autoimmune diseases (e.g. rheumatoid arthritis, scleroderma);
iii. blood vessel diseases (e.g. arteritis/Buerger disease);
iv. cardiac diseases (e.g. myocardial infarction, chronic heart failure);
v. respiratory system diseases (e.g. chronic obstructive pulmonary diseases, idiopathic pulmonary fibrosis);
vi. skeletal system diseases (e.g. bone regeneration, avascular necrosis, osteomyelitis, cartilage repair, tendon repair, muscular dystrophies);
vii. gastrointestinal tract diseases (e.g. fistulae, ulcers, esophageal stricture, cirrhosis, incontinence, Crohn's disease);
viii. kidney disease (e.g. nephropathy);
ix. urinary tract (e.g. incontinence);
x. skin diseases (e.g. foot ulcers, epidermolysis bullosa, pemphigus, diabetic ulcers, static venous ulcers, chronic pressure ulcers);
xi. ageing associated diseases;
xii. peripheral nerve and skeletal muscle diseases (e.g. chronic inflammatory demyelinating polyradicularneuropathy, Guillan Barre syndrome, muscular dystrophies);
xiii. diseases of the central nervous system (e.g. neurodegenerative diseases such as demyelinating diseases (multiple sclerosis), Alzheimer's disease, Parkinson's disease, bulbospinal atrophy, cerebral stroke, spinal ischemia, disease of the autonomic nervous system as multiple systemic atrophy);
xiv. eye diseases (e.g. retinopathies [aged macular degeneration, diabetic retinopathy, arteriosclerotic retinopathy), optic neuritis);
xv. diseases of the endocrine system (e.g. diabetes and its complications: [vascular disorders, neuropathies, chronic ulcers, nephropathy]); and
xvi. dental and oral diseases (e.g. dental pulp related diseases, periodontal diseases, alveolar bone defects, oral mucosa ulceration caused by immune diseases).
Each possibility represents a separate embodiment of the present invention.

According to a specific embodiment, the disorder is diabetic wound.

According to some embodiments, the disorder is a cosmetic disorder.

According to some embodiments, the method of treating involves tissue remodeling, tissue repair or tissue regeneration and comprises administering to a subject in need thereof a cell-free composition comprising hOMSC-derived secretome, said composition may be a pharmaceutical or a cosmetic composition.

According to some embodiments, a method for promoting or accelerating diabetic wound healing is provided comprising administering to a subject in need thereof a cell-free composition comprising hOMSC-derived secretome.

According to yet another aspect, the present invention provides a method for tissue repair and regeneration comprising administering at least one cell-free composition comprising substances secreted from human oral mucosa stem cells (hOMSC) according to the invention.

According to some embodiments, the repair or regeneration methods are of organs and tissues that were totally or partially destroyed by mechanical trauma, chemical injuries, radiation, and heat or any other type of iatrogenic injuries. Examples of such injuries include but are not limited to: contusion of the central nervous system, spinal injuries, section of the spinal cord, peripheral nerve crush or section, burns, neuropathy and cardiopathy due to chemotherapy, bone fractures, tendon and ligament rupture. Each possibility represents a separate embodiment of the present invention.

According to some embodiments tissue repair or regeneration is associated with a condition, disease or disorder selected from the group consisting of: wound healing, degenerative diseases, congenital defects, aging related defects, and iatrogenic defects.

According to a specific embodiment the stem cells are either allogeneic or autologous.

The compositions of the present invention may be administered to a subject in need thereof, via any suitable route of administration, including but not limited to topically, subcutaneously, intramuscularly, intravenously, intra-arterially, intraarticularly, intralesionally, intratumorally or parenterally. According to some embodiments, for wound healing, topical administration may be used. Pharmaceutical and cosmetic compositions according to the present invention are thus formulated to fit the specific route of administration used. For example, for topical administration, the compositions may be formulated as creams, foams, gels, lotions, and ointments, using methods known in the art.

According to some embodiments, the composition is administered locally to the injured tissue.

Compositions according to the present invention, comprising hOMSC-derived secretome, may be administered to the injured tissue, according to any treatment regimen. For example, the compositions may be administered once or multiple times to the same or to different locations.

According to some embodiments, the cell-free compositions of the present invention, comprising hOMSC-derived secretome, are administered to a subject in need thereof, as part of a treatment regimen comprising at least one additional pharmaceutical or cosmetic agent or treatment.

Essentially all of the uses known or envisioned in the prior art for stem cells can be accomplished with the secretomes of the present invention derived from hOMSC. These uses include prophylactic and therapeutic techniques.

Further embodiments and the full scope of applicability of the present invention will become apparent from the detailed description given hereinafter. However, it should be understood that the detailed description and specific examples, while indicating preferred embodiments of the invention, are given by way of illustration only since various changes and modifications within the spirit and scope of the invention with become apparent to those skilled in the art from this detailed description.

### BRIEF DESCRIPTION OF THE FIGURES

FIG. 1. Comparison between the stem cell marker profile of hOMSC and foreskin stem cells (hSkin). The results are represented as −ΔCt (cycle threshold) values relative to the house-keeping gene GAPDH. A higher negative values means a lower level of expression.

FIG. 2 Relative differences in protein expression between hOMSC and hSkin SC secretomes from immunofluorescent staining with antibodies against pluripotency- and neural crest-associated stem cell markers.

FIG. 3. Ratio of expression of selected makers in hOMSC and hSkin SC.

FIG. 4. Protein expression of the secretome of mesenchymal stem (stromal) cells obtained from young human bone marrow. Data taken from Park et al., International Journal of Stem Cells, 2009.

FIG. 5. The upper left panel shows the donut-shaped ring sutured to the dorsal skin of a diabetic db/db mouse before wounding. The upper right panel depicts the site immediately after wounding. The lower left panel illustrates the site of intradermal injections. The lower right panel shows the histology or the excised skin.

FIGS. 6A and 6B. Quantitative (6A) and representative qualitative photographs (6B) that illustrate the rate of diabetic wound healing in groups of db/db diabetic mice treated with either hOMSC or hSkin SC or with PBS vehicle (Untreated).

FIG. 7. Quantitative illustration of the rate of diabetic wound healing in groups of: db/db diabetic mice treated with hOMSC, db/db diabetic untreated mice, or wild-type (WT)-untreated mice.

FIG. 8. Average time required for complete wound closure in WT-untreated mice, WT-hOMSC-treated mice, db/db-untreated mice, db/db-hOMSC treated mice and db/db hADSC (human adipose tissue-derived stem cells) treated mice. The calculated t-Test p values are: WT-untreated vs. db-hOMSC=0.19533; db-hOMSC treated vs. db-untreated=5.34E-6; db-hOMSC treated vs. db-hADSC treated=0.00044; db-untreated vs. db-hADSC treated=0.48983; WT-untreated vs. db-hOMSC treated=1.9933E-5; WT-untreated vs. db-untreated=2.262E-5.

FIG. 9. Quantitative illustration of the rate of diabetic wound healing in groups of db/db diabetic mice treated with: hOMSC, hSkin SC, hADCS or with PBS vehicle (Untreated).

FIG. 10. Quantitative illustration of the rate of diabetic wound healing in groups of db/db diabetic mice treated with:hOMSC, hOMSC-derived cell-free secretome, hSkin stem cells or with PBS vehicle (Untreated).

FIG. 11. 369 hOMSC secretome proteins identified by mass spectrophotometry, and their average relative abundance (intensity).

FIG. 12. 294 hOMSC secretome proteins that are common to the 1534 proteins listed for the secretomes derived from either BMSC. ASC or DPSC.

### DETAILED DESCRIPTION OF THE INVENTION

The present invention provides secretomes of adult stem cells from human oral mucosa for treatment and prevention of diseases and disorders.

hOMSC are a neural crest (NC)-derived stem cell type, which co-express the pluripotency markers Oct4, Nanog and Sox2 as well as the NC—SC markers, Snail, Slug, Sox10, Twist and Notch 1 in developing colonies (Marynka-Kalmani et al. 2010; Widera et al. 2009). The NC is a transient neuroectodermal structure of the vertebrate embryo. During its embryonic existence it gives rise to migratory multipotent stem cells that populate various primordial tissues where they differentiate into neural lineages and or lineages with a mesenchymal phenotype termed ectomesenchyme or mesectoderm. Some of these NC—SC remain in a relative undifferentiated state in the adult, with a predisposition for neural differentiation even in tissues of mesenchymal origin such as dermis and bone marrow.

Typical whole adult populations contain low amount of stem cells and therefore expansion and isolation of stem cells are laborious, long and usually not efficient. It was demonstrated that primary whole population and expanded whole cell population derived from the lamina propria of the oral mucosa consists mainly (more than 80%) of naïve stem cells. High proportions (80-90%) of cell populations obtained from the oral mucosa of three different donors were shown to express mesenchymal stem cells markers. The study of Marynka-Kalmani et al. 2010 (ibid) has proved that trillions of hOMSC are cost-effectively and reproducibly generated from a biopsy of 3-4×2×1 mm that is obtained with negligible morbidity.

A typical isolation method of stem cells from a solid tissue for clinical utilization comprises releasing the cells from the extracellular matrix by enzymatic digestion or by explantation; expanding primary whole population in order to obtain sufficiently large populations; and isolation of stem cells from the whole populations.

The quality and quantity of the isolated stem cells population form the lamina propria oral mucosa is largely unaffected by aging and can be expanded in vitro without losing its pluripotency and is therefore a safe and reliable source for secretomes to be re-administered to a subject in need thereof to effectively achieve tissue regeneration and other therapeutic processes.

### Definitions

Oral Mucosa is the mucosal lining the oral cavity, namely: the cheeks and the alveolar ridge including the gingiva and the palate, the tongue, the floor of the mouth and the oral part of the lips. Oral mucosa consists of an epithelial tissue of ectodermal origin and the lamina propria (LP) which is a connective tissue of ectomesenchymal origin. Similar to the ectomesenchymal origin of connective tissues in the oral cavity, cells of the oral mucosa lamina propria (OMLP) originate from the embryonic ectodermal neural crest. Wounds in human oral mucosa heal mainly by regeneration. The rate of healing is faster than that in the skin or other connective tissues and seems to be affected negligibly by age and gender (Szpaderska, A. M., et al., J Dent Res, 2003, 82, 621-626).

“Stem cells” (SC) are undifferentiated cells, which can give rise to a succession of mature functional cells.

“Embryonic stem (ES) cells” are cells derived from the inner cell mass of the embryonic blastocysts that are pluripotent, thus possessing the capability of developing into any organ or tissue type or, at least potentially, into a complete embryo.

“Adult stem cells” are post-natal stem cells derived from tissues, organs or blood of an organism after its birth.

“Pluripotent stem cells” are stem cells capable of generating the three embryonic cell layers and their derivatives cell lineages and tissues;

“Multipotent stem cells” are stem cells capable of forming multiple cell lineages that constitutes an entire tissue or organ;

Secretome according to the present invention is a composition comprising soluble and insoluble substances in their various forms that are secreted or released into the culture medium from human oral mucosa derived stem cells. Amongst others these substances include:
2. Soluble molecules as:
   a. Proteins
   b. Peptides
   c. Hormones
   d. various DNA and RNA species
   e. oligomers of nucleic acids
   f. other molecules with a molecular weight higher than 1,000 Daltons
3. Extracellular vesicles that contain:
   a. Proteins: growth factors, cytokines, hormones, cell surface receptors, cytosolic and nuclear proteins, metabolic enzymes, receptor ligands, adhesion proteins, endosome associated proteins, tetraspanins, lipid raft associated proteins, antigens, etc.
   b. RNA species: mRNA, miRNA, tRNA, rRNA, siRNA, and lncRNA and possible other RNA species
   c. DNAs: mitochondrial DNA (mtDNA), single stranded DNA (ssDNA), double stranded DNA (dsDNA)
   d. Lipids: cholesterol, sphingomyelin, hexosylcermides and others
   e. Lectins, glycans, proteoglycans, glycoproteins.

Extracellular Vesicles are membrane bound particles that carry cargo of soluble and insoluble substances mentioned above. The term “Extracellular Vesicles” refers a group of secreted or shedded vesicles of various species. These are divided in the following subtypes (Xu et al. JIC 2016):
1. Microvesicles or Shed Microvesicles; size range—50-1500 nm
2. Exosomes; size range—30-120 nm
3. Vesicles; size range <500 nm

Culture medium or expansion medium is the medium in which the hOMSC are cultured and expanded. Culture/expansion medium according to some embodiments of the present invention comprises at least one of the following components: low glucose Dulbecco's modified Eagle's medium (LGDMEM), streptomycin, penicillin, gentamycin, amphotericin B, glutamine and serum, for example fetal calf serum (FCS).

According to some embodiments, the culture expansion medium comprises low LGDMEM supplemented with 100 μg/ml streptomycin, 100 U/ml penicillin, (Biological Industries, Beit-Haemek, Israel), glutamine 2 mM (Invitrogen) and 10% fetal calf serum (FCS, Gibco).

Basal medium is the culture medium without serum.

Conditioned medium according to the present invention refers to the medium collected from hOMSC cultures comprising hOMSC-derived substances secreted or released into the medium in which they are grown or maintained.

The conditioned medium comprising the hOMSC secretome may optionally be concentrated using methods known in the art to increase the concentration of the secretome constituents and then preserved, for example in a frozen state. Alternatively, the conditioned medium can be lyophilized and the secretome preserved as a frozen powder and reconstituted in water for injection or saline or other known in the art solution for injection.

The concentrated conditioned medium containing the secretome or the lyophilized secretome, may be supplemented with any additive or preservative known in the art and then stored, according to some embodiments in a condition and temperature to maintain the substances in their native and effective form.

The secretomes of the present invention may be admixed with at least one excipient or carrier that is pharmaceutically acceptable and compatible with the secretome' ingredients as is well known. Suitable excipients are, for example, water, saline, phosphate buffered saline (PBS), Plasma Lyte, dextrose, glycerol, ethanol, polyethylene glycol, mineralized excipients such as hydroxyapatite particles and tricalciumphosphate putty or particles, and combinations thereof. Excipient and carriers may also include extracellular matrix components such as proteins (collagens, elastin, attachment proteins e.g. fibronectin, vitronectin, albumin, etc.); glycoproteins (osteopontin, bone sialoproteins, thrombonspondin, tenascin, etc).; proteoglycans and glycoseaminoglycans (hyaluronic acid, chondroitin sulfate, dermatan sulfate, heparan sulfate etc.). Other suitable excipients and carriers are well known to those skilled in the art.

In addition, if desired, the composition can contain minor amounts of auxiliary substances such as emulsifying agents, pH buffering agents etc.

The term “treatment” as used herein refers to both therapeutic treatment and prophylactic or preventative measures. Those in need of treatment include those already with the disorder as well as those in which the disorder is to be prevented.

The term “administering” or “administration of” a composition to a subject can be carried out using one of a variety of methods known to those skilled in the art. For example, a composition can be administered enterally or parenterally. Enterally refers to administration via the gastrointestinal tract including per os, sublingually or rectally. Parenteral administration includes administration intravenously, intradermally, intramuscularly, intraperitoneally, subcutaneously, ocularly, sublingually, intranasally, by inhalation, intraspinally, intracerebrally, and transdermally (by absorption, e.g., through a skin duct). A composition can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the appended claims.

### Examples

The results described below were obtained in part from secretomes of cell populations derived from the lamina propria (not including the epithelial part) of the human gingiva which is an integral part of the oral mucosa lining the oral cavity. hOMSC isolated from the palate and alveolar mucosa exhibit the same properties.

hOMSC Isolation and Culture

hOMSC were obtained from oral mucosa biopsies particularly of gingival origin from donors aged 25-80 years as described above and in WO 2008/132722.

Briefly, gingival or alveolar mucosa biopsies 3-4×2×1 mm were minced and explants were cultured in 25 cm² tissue culture flasks in low glucose Dulbecco's modified Eagle's medium (LGDMEM) supplemented with 100 μg/ml streptomycin, 100 U/ml penicillin, (Biological Industries, Beit-Haemek, Israel), glutamine 2 mM (Invitrogen) and 10% fetal calf serum (FCS) (Gibco) as described by Marinka-Kalmani et al 2010 (ibid). This medium is referred as culture medium or expansion medium. In some cases the streptomycin and penicillin are replaced with gentamycin, and amphotericin B is also included in the expansion medium.

### Secretome Generation

Cultures of hOMSC having a cumulative population doubling between 5-80 that are expanded in expansion medium are used for generating the hOMSC secretome. The expansion medium is removed and the cultures are washed exhaustively with PBS and then either basal medium or LGDMEM is added. After 24-120 hours the medium is collected and centrifuged to remove any dead cells. The supernatant contains the secretome.

The concentration of the secretome components can be increased by concentrating the supernatant using devises and methods known in the art. As shown herein, according to some embodiments, a concentration ratio ranging from 1.1-10,000 folds can be envisaged to be useful and effective for achieving a desired therapeutic effect.

Array analyses at the protein and molecular levels indicate that hOMSC secretome has a unique composition signature that has not been found before in stem cell secretomes derived from other sources, including skin-derived stem cells and bone marrow-derived stem cells.

The secretome composition may be changed by subjecting hOMSC to various culture conditions and stimulation. Examples of such culture stimulation and conditions include but are not limited to:
Chemical: e.g. hypoxia, hyperoxia, chemical drugs, various classes of chemical stimulators or inhibitors or various pathways, hypo or hyper-ionic concentration as for example Ca⁺⁺ and/or glucose, various chemical drugs as statins, bisphosphonate, etc;
Physical: e.g. ultrasound, mechanical vibration, electrical stimulation, continuous or intermittent strain, light, radiation;
Substrate: e.g. attachment proteins, 3 dimensional matrices, beads for suspension cell culture;
Biologics: e.g. growth factors, cytokines, hormone stimulation, differentiation factors, DNA and RNA species, genetic manipulations.

### Example 1. Comparison of Stem Cell Markers of hOMSC to Those or Other Sources

hOMSC were obtained as described previously (Marynka-Kalmani et al. 2010 and WO 2008/132722). Foreskin SC (hSkin SC) were isolated by enzymatic digestion from the foreskin of 8-day old infants. Both cell types were grown in T-75 cell flasks in low glucose DMEM supplemented with essential amino acids antibiotics and fetal calf serum.

Stem cell markers of hOMSC and foreskin stem cells (hSkin SC), were assessed by RT-PCR and immunochemistry (Marinka-Kalmani et al 2010, ibid and unpublished data).

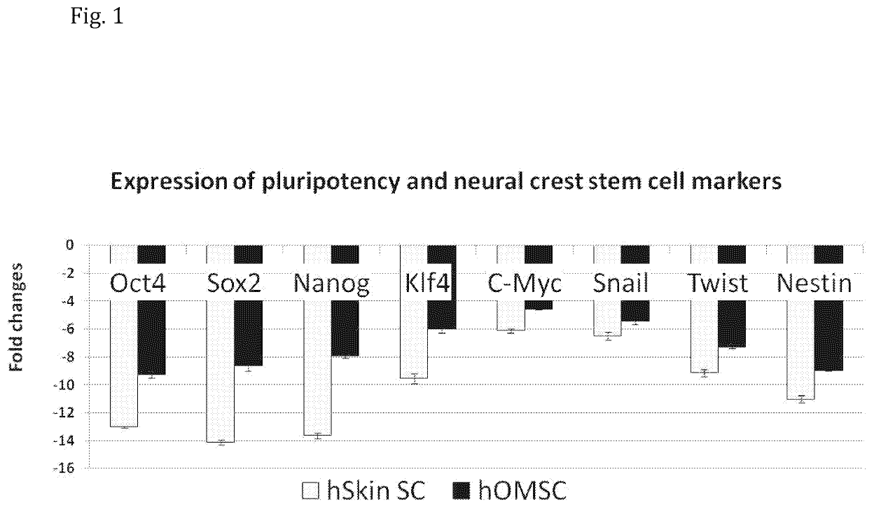

As shown in FIG. 1, hOMSC are endowed with a higher expression of pluripotency and neural crest associated markers than hSkin SC.

The markers OCT4, SOX2, and NANOG are characteristic pluripotency associated markers; c-MYC and KLF4 are both pluripotency associated and early neural crest markers; and SNAIL is a characteristic neural crest stem cell marker.

The molecular data was confirmed at the protein level by immunofluorescence staining with antibodies against pluripotency- and neural crest-associated stem cell markers. indicating that the markers NANOG, SOX2, C-MYC, KLF4 and SNAIL are more abundant in hOMSC than in the hSkin SC. It was also found that the staining in hOMSC is restricted to the nuclei suggesting functional activity of these transcription factors. It is therefore concluded that there is clear higher expression of these markers in hOMSC compared to hSkin Sc.

### Example 2. Global Analysis of hOMSC Secretome

The unique signature of the hOMSC secretome is confirmed by determining its protein and nucleic acid content. Three different method are used to obtain a broad spectrum of hOMSC secretome components: protein arrays, mass spectrophotometry (MS) and microRNA (miRNA) characterization.

hOMSC are generated and expanded in expansion medium as described above. The protein profile in the condition medium was assessed by mass spectrophotometry and by commercially available protein array kits. The sequence of RNA specifies contained within the hOMSC secretome is performed using methods known in art, to determine the genetic cargo of hOMSC secretome.

### Protein Profile

The protein content of hOMSC secretome was analyzed by MS and protein array.

MS analysis: Four secretomes, from 4 different hOMSC cultures, each derived from a separate donor, are prepared as described above. 0.1 ml samples are digested by trypsin, analyzed by LC-MS/MS on Q exactive plus (Thermo Fisher) and analyzed by Discoverer software version 1.4 against the human and bovine uniprot database (for fetal calf serum). The identified proteins are filtered for false discovery rates (FDRs)<0.01 in the peptide- and protein-level using the target-decoy strategy.

The proteins are filtered to eliminate the common contaminants and single peptide identifications. Semi quantitation was done by calculating the peak area of each peptide. The area of the protein is the average of the three most intense peptides from each protein.

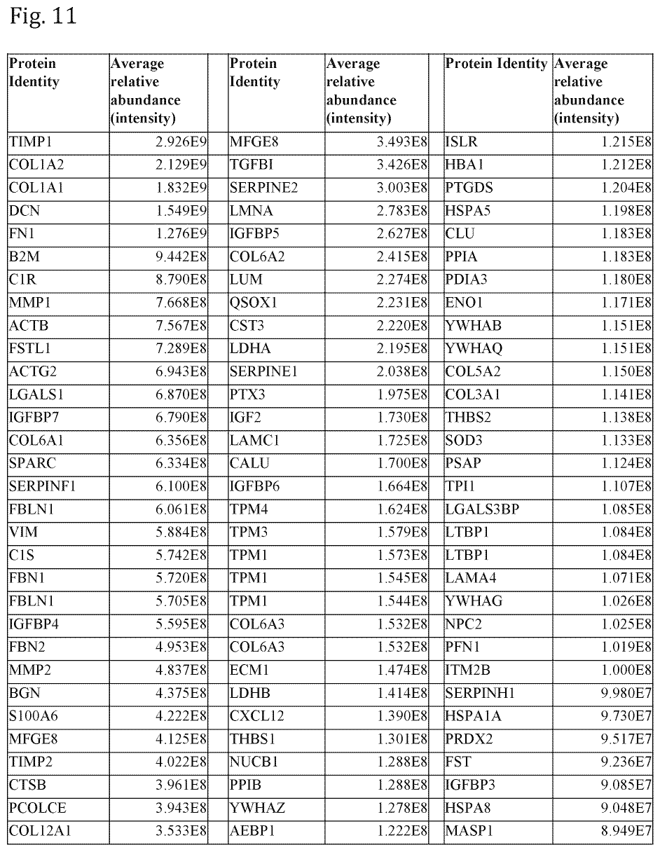

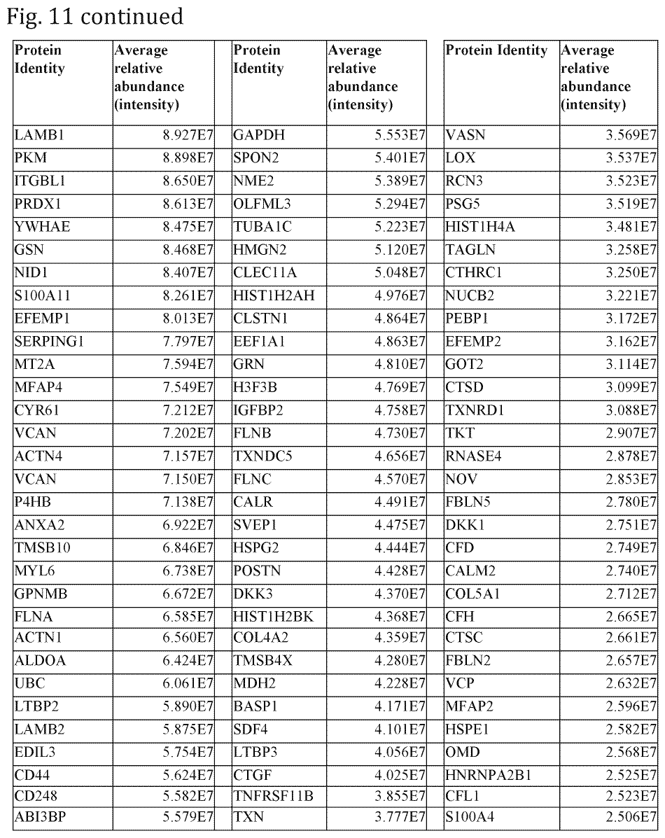

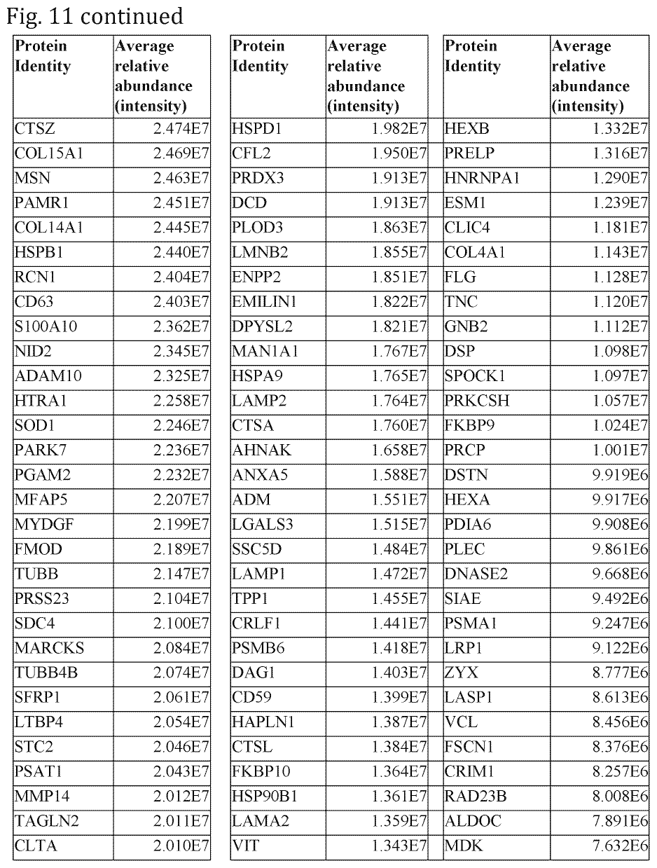

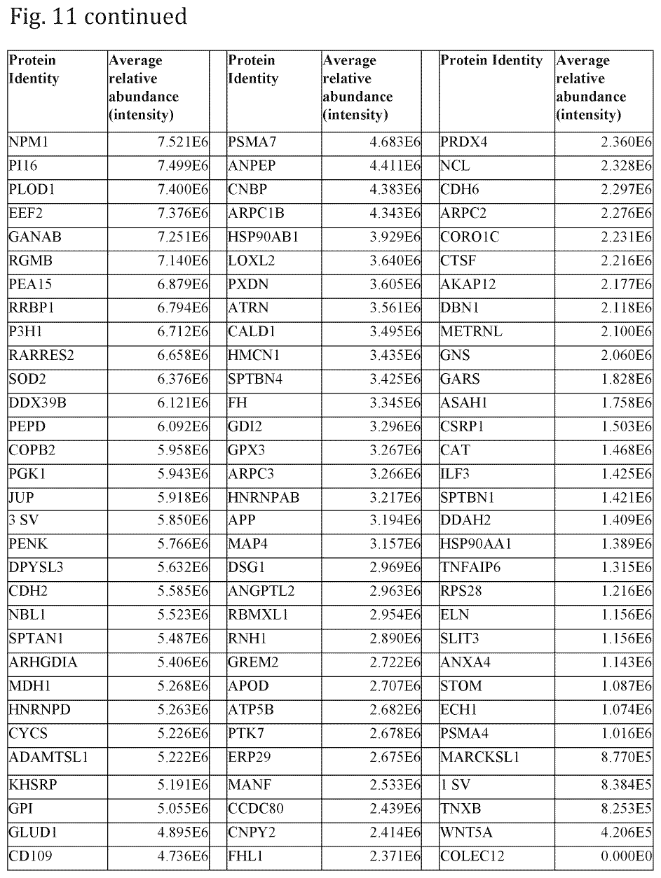

A total of 369 proteins were identified within hOMSC secretome (FIG. 11), including extracellular matrix proteins, glycoproteins, protein receptors, proteolytic enzymes for extracellular matrix proteins and proteolytic enzyme inhibitors, proteins involved in metabolism, proteins involved in stress responses, such as heat shock proteins, nuclear proteins, proteins involved in tissue development and repair, integral cell membrane proteins as integrins and clusters of differentiation (CD) proteins including exosomal markers CD63, immune-modulatory proteins and other clusters of proteins.

Noteworthy are a cluster of 13 proteins that are involved in the homeostasis, protection and repair of the nervous system, detailed in Table 1:

TABLE 1
hOMSC secretome proteins involved in the homeostasis of the nervous system.

| | | | Relative |
|---|---|---|---|
| Accession No. | Protein Description | Gene Name | Expression |
| P01034 | Cystatin-C OS = Homo sapiens GN = CST3 PE = | CST3 | 2.220E8 |
| | 1 SV = 1 − [CYTC_HUMAN] | | |
| P09382 | Galectin-1 OS = Homo sapiens GN = LGALS1 PE = | LGALS1 | 6.870E8 |
| | 1 SV = 2 − [LEG1_HUMAN] | | |
| P07093 | Glia-derived nexin OS = Homo sapiens | SERPINE2 | 3.003E8 |
| | GN = SERPINE2 PE = 1 SV = 1 − [GDN_HUMAN] | | |
| P01344 | Insulin-like growth factor II OS = Homo sapiens | IGF2 | 1.730E8 |
| | GN = IGF2 PE = 1 SV = 1 − [IGF2_HUMAN] | | |
| E7EV71 | Latent-transforming growth factor beta-binding | LTBP1 | 1.084E8 |
| | protein 1 OS = Homo sapiens GN = LTBP1 PE = | | |
| | 1 SV = 2 - [E7EV71_HUMAN] | | |
| G3V511 | Latent-transforming growth factor beta-binding | LTBP2 | 5.890E7 |
| | protein 2 OS = Homo sapiens GN = LTBP2 PE = | | |
| | 1 SV = 1 − [G3V511_HUMAN] | | |
| H0YC99 | Latent-transforming growth factor beta-binding | LTBP3 | 4.056E7 |
| | protein 3 (Fragment) OS = Homo sapiens GN = LTBP3 | | |
| | PE = 1 SV = 1 − [H0YC99_HUMAN] | | |
| A0A0C4DH07 | Latent-transforming growth factor beta-binding | LTBP4 | 2.054E7 |
| | protein 4 OS = Homo sapiens GN = LTBP4 PE = | | |
| | 1 SV = 1 − [A0A0C4DH07_HUMAN] | | |
| P55145 | Mesencephalic astrocyte-derived neurotrophic factor | MANF | 2.533E6 |
| | OS = Homo sapiens GN = MANF PE = 1 SV = 3 − | | |
| | [MANF_HUMAN] | | |
| Q09666 | Neuroblast differentiation-associated protein | AHNAK | 1.658E7 |
| | AHNAK OS = Homo sapiens GN = AHNAK PE = | | |
| | 1 SV = 2 − [AHNK_HUMAN] | | |
| P36955 | Pigment epithelium-derived factor OS = Homo sapiens | SERPINF1 | 6.100E8 |
| | GN = SERPINF1 PE = 1 SV = 4 − [PEDF_HUMAN] | | |
| P48061 | Stromal cell-derived factor 1 OS = Homo sapiens | CXCL12 | 1.390E8 |
| | GN = CXCL12 PE = 1 SV = 1 − [SDF1_HUMAN] | | |
| P00441 | Superoxide dismutase [Cu-Zn] OS = Homo sapiens | SOD1 | 2.246E7 |
| | GN = SOD1 PE = 1 SV = 2 − [SODC_HUMAN] | | |

Of special interest are the existence of SOD1 and mesencephalic astrocyte derived-neurotrophic factor (MANF) in hOMSC secretome. These proteins suppress the intracellular stress, which is a landmark of neurodegenerative diseases and a cause of cell death. Moreover, MANF has been shown to be efficient in the treatment of Parkinson Disease in animal models (Voutilainen et al. 2015).

The protein composition of hOMSC secretome as determined by MS is unique. When hOMSC secretome is compared to that of mesenchymal stem cells derived from human bone marrow (BMSC), adipose tissue (ASC) or dental pulp (DPSC) (Tachida et al. 2015), it is found that hOMSC secretome contains 75 proteins that are not detected in any of the secretomes.

The 75 unique proteins identified are listed in Table 2:

TABLE 2
Unique hOMSC secretome proteins.

| Protein | Accession No. | Protein | Accession No. | Protein | Accession No. |
|---|---|---|---|---|---|
| 1 SV | E7ENT3 | DSTN | F6RFD5 | MFAP4 | P55083 |
| 3 SV | HOYAE9 | ECH1 | M0R248 | MMP1 | P03956 |
| ACTG2 | P63267 | EDIL3 | O43854 | MMP14 | P50281 |
| ADAM10 | 014672 | EFEMP1 | A0A0UIRQV3 | MT2A | P02795 |
| ADAMTSL1 | Q8N6G6 | ELN | E7ETP7 | NBL1 | A3KFI5 |
| ADM | EP9L83 | FLG | P20930 | OMD | Q99983 |
| ANXA4 | Q6P452 | GNB2 | C9JIS1 | PFN1 | P07737 |
| APOD | P05090 | GREM2 | G9H772 | PI16 | Q6UXB8 |
| CALM2 | P0DP24 | H3F3B | K7EMV3 | PSG5 | E7EQY3 |
| CD109 | Q6YHK3 | HBA1 | P69905 | PSMB6 | P28072 |
| CD59 | E9PNW4 | HIST1H2AH | Q96KK5 | PTGDS | P41222 |
| CDH6 | DHRF86 | HIST1H2BK | O60814 | RARRES2 | Q99969 |
| CFD | P00746 | HIST1H4A | P62805 | SLIT3 | A0A0A0MSC8 |
| COL15A1 | A0A087X0K0 | HMGN2 | P05204 | SPOCK1 | Q08629 |
| COL1A2 | A0A087WTA8 | HNRNPAB | D6R9P3 | SPTBN4 | M0QZQ3 |
| COLEC12 | Q5KU26 | HSP90AA1 | P07900 | STOM | P27105 |
| CTHRC1 | Q96CG8 | HSPA1A | P0DMV8 | TMSB10 | P63313 |
| CTSC | H0YCY8 | HSPG2 | P98160 | TMSB4X | P62328 |
| CTSL | P07711 | IGFBP5 | P24593 | TNFAIP6 | P98066 |
| CXCL12 | P48061 | JUP | P14923 | TNXB | A0A087WWA5 |
| DCD | P81605 | KHSRP | M0R0I5 | TPI1 | P60174 |
| DDAH2 | O95865 | LDHA | P00338 | TUBA1C | Q9BQE3 |
| DKK1 | O94907 | LMNB2 | Q03252 | UBC | F5H6Q2 |
| DSG1 | Q02413 | LTBP4 | A0A0C4DH07 | VIT | Q6UXI7 |
| DSP | P15924 | MAN1A1 | P33908 | WNT5A | P41221 |

The protein CXCL12, which is unique to hOMSC secretome, is known to be instrumental in stem cell recruitment to injured organs and promote proliferation and migration of neural progenitor cells (Wu et al. 2009). This protein is highly abundant in hOMSC secretome being ranked 58 out of 369 proteins, namely in the upper 20% of the detected proteins.

Peripheral tissues under stress caused by disease or injury secrete CXCL12 to recruit endothelial progenitors and mesenchymal stem (stromal) cells from the bone marrow. This process is substantially depressed in injured diabetic tissues (Rodrigues et al. 2015, Tepper et al. 2010). Administration of the hOMSC secretome that contains CXCL12 as a main trophic factor at the injured tissue, enhances wound healing in general and in diabetic individuals in particular.

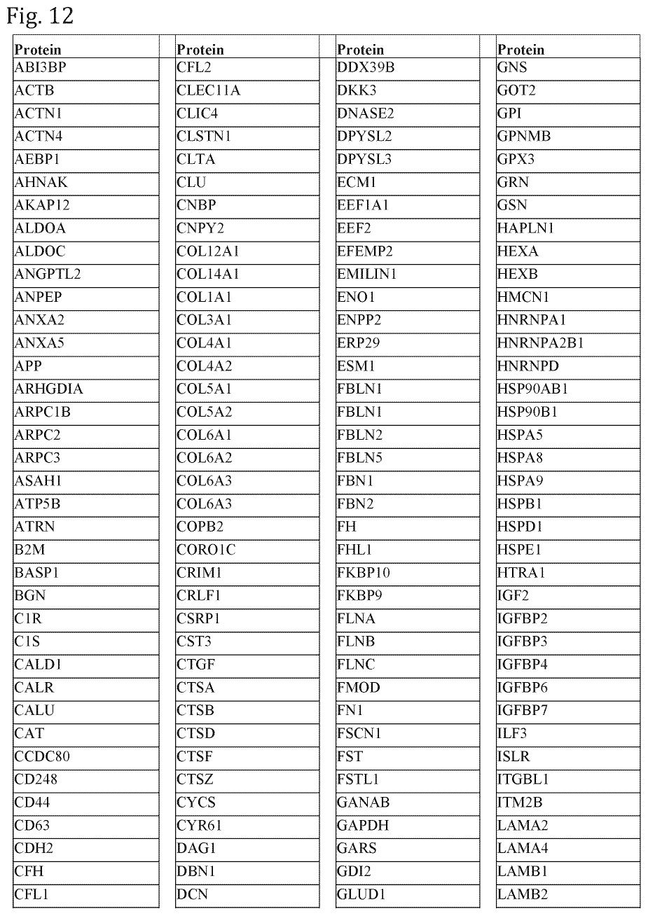

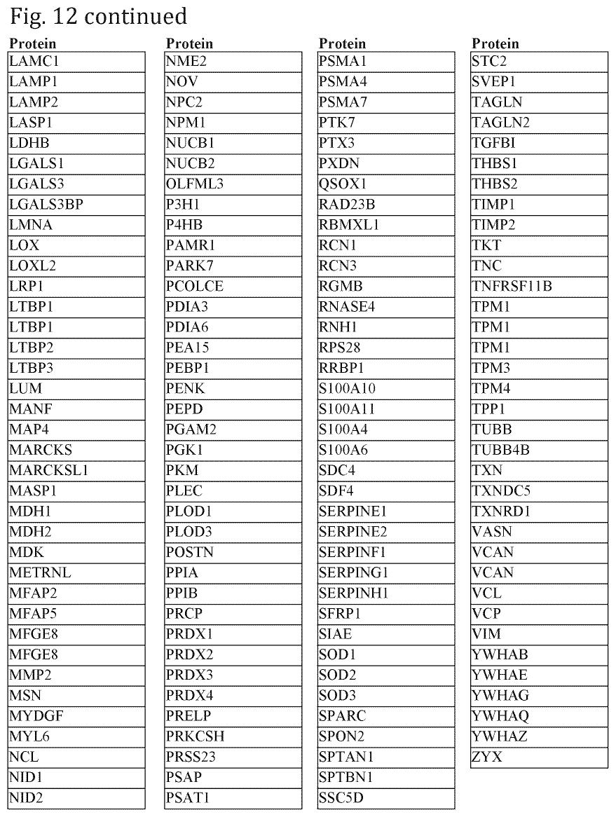

As shown in FIG. 12, a total of 294 hOMSC secretome proteins are common to the 1534 proteins listed for the secretomes derived from either BMSC. ASC or DPSC. However, the unique signature of a secretome is determined not only by its components but also by the relative abundance of these components. For example, Insulin Growth factor 2 (IGF2), that belongs to the insulin family of growth factors and has pleiotropic functions in tissue homeostasis and repair is a major component of hOMSC secretome, but is barely detected in BMSC and undetected in ASC and DPSC.

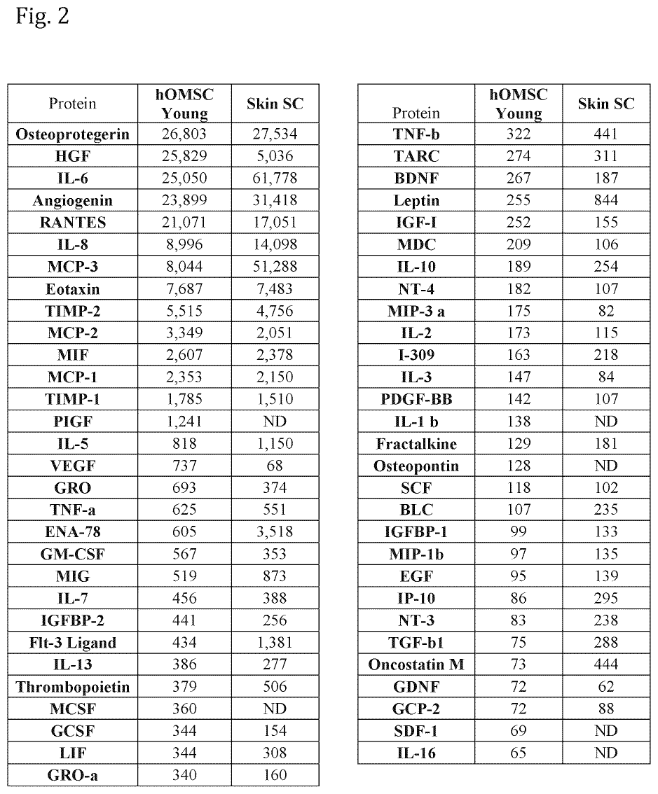

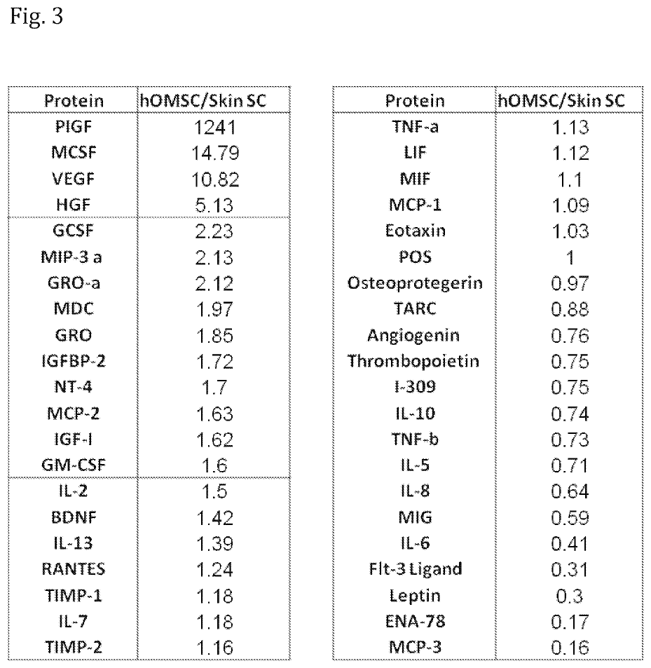

Protein array: The protein component of the secretome of hOMSC was further analyzed and compared to that of hSkin SC by a protein array kit of 80 proteins (RayBio® G-Series Cytokine Array, RayBiotech, Inc, USA). Protein analysis revealed the differences in the secretion of at least 21 proteins that were either over- or under-expressed in hOMSC secretome compared to that of hSkin SC (FIGS. 2 and 3). Notable are the proteins P1GF, MSCF, VEGF and HGF for the over abundant cytokines and the proteins leptin, ENA-78 and MCP-3 for the under abundant in hOMSC compared with hSkin SC.

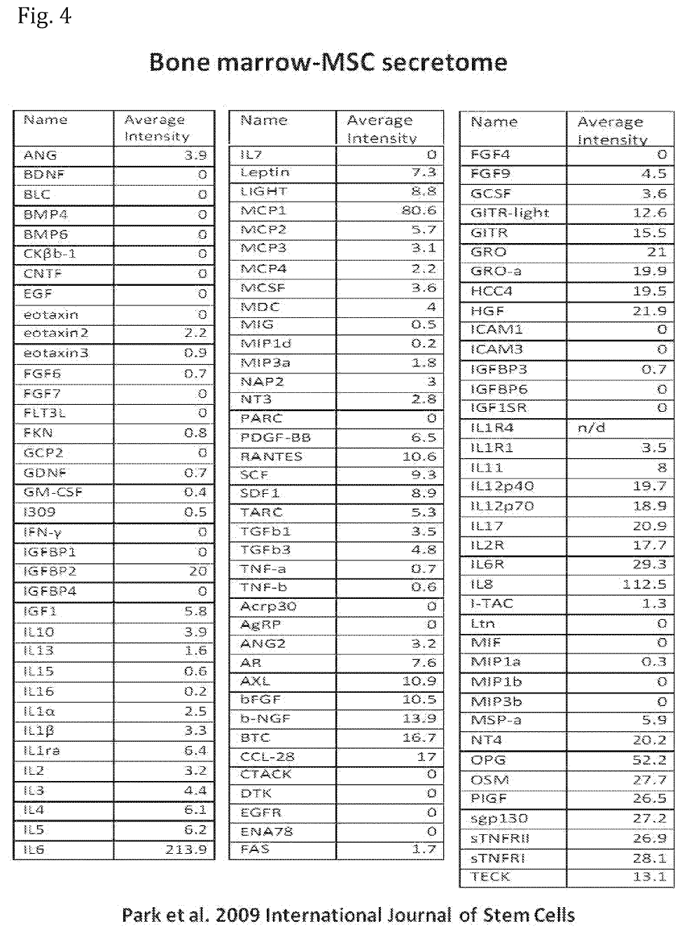

The secretome of hOMSC and hSkin SC was further compared to that published for human mesenchymal stem cells derived from young human bone marrow that used the same antibody array kit that was used to determine the secretome of hOMSC and hSkin SC described above (Park et al. 2009). Considering the lower detection limit at the value of 50, comparison of the tables in FIGS. 2 and 4 shows that growth factors and neurotrophic agents as P1GF, EGF, SDF-1, BDNF, GDNF, IGF1, Angiogenin and many other are undetectable in the secretome of bone marrow derived mesenchymal stem cells but are expressed in hOMSC.

Some of the factors and agents, such as, for example, HGF, are highly expressed in hOMSC. The differences between hOMSC secretome and that of skin SC is shown in FIG. 3, which illustrates the proportions between the quantities of various chemokines within hOMSC secretome and skin SC one. The data presented in FIGS. 1-4 clearly demonstrate that the 3 stem cell types have a different secretory profile as of the proteins tested.

MicroRNA analysis: Conditioned medium was collected from three different hOMSC cultures, each derived from a different donor, as described above for the preparation of the secretome, but without performing the concentration step. The conditioned medium was centrifuged for 4 minutes at 12000 G at 4° C., total miRNA was extracted according to the procedure below:
1. 7 ml of Trizol™ reagent is added to 10 ml of sample and vortexed. Then the samples are left to stand for 5 minutes at room temperature.
2. 1.4 ml of chloroform is added and the sample shaken vigorously for 15 seconds.
3. Samples are centrifuged at 15,300G at 4° C., for 15 minutes.
4. The upper aqueous phase is transferred to a new tube, carefully avoiding the interphase.
5. miRNA is isolated using mirVana™ PARIS miRNA Isolation Kit (Ambion®) according the manufacturer's protocol. The mirVana™ kit utilizes two sequential GFFs.
   miRNA is eluted in 50 μl RNase-free water.

The concentration and purity of miRNA was assessed using NanoDrop™ light spectrophotometer (NanodropTechnologies, Wilmington, DE, USA). The wavelength dependent extinction coefficient represents the microcomponent of all RNA in solution as shown the Table 3:

TABLE 3
Wavelength-dependent
extinction coefficient values

| | | | Extinction |
|---|---|---|---|
| Sample | | Volume | Coefficient |
| # | ng/μl | (μl) | at 260/280 nm |
| 1 | 38.5 | 17 | 2.11 |
| 2 | 30.9 | 17 | 2.15 |
| 3 | 40.7 | 17 | 2.1 |

Characterization of microRNAs Expression

miRNA expression profiling was performed using the nCounter miRNA Expression Assay (described in https://www.nanostring.com), that provides a method for detecting 800 miRNAs without the use of reverse transcription or amplification by using molecular barcodes called nCounter Reporter Probes. All data analysis and normalization were performed using the nSolver™ Software Analysis (complimentary download from NanoString Technologies) in which specific miRNA counts are normalized to a selection of stably expressed miRNAs based on CVS statistics calculated across all experimental samples or with the use of the Spike-in controls.

The normalized data shown in Table 4 indicates 39 miRNAs that are expressed in the 3 hOMSC secretomes, each derived from a different donor. Each of this miRNAs has a relative expression value higher than 20, which is an accepted lower threshold for miRNA detection when the methodology described above is used.

TABLE 4
miRNAs having a relative value >20, identified in 3 unique hOMSC secretomes.

| | Expressed | |
|---|---|---|
| miRNA name | also in | Reference |
| hsa-miR-4454 + hsa-miR-7975 | — | MIMAT0018976 |
| hsa-miR-23a-3p | — | MIMAT0000078 |
| hsa-let-7b-5p | — | MIMAT0000063 |
| hsa-miR-612 | — | MIMAT0003280 |
| hsa-miR-125b-5p | BMSC | MIMAT0000423 |
| hsa-miR-3144-3p | — | MIMAT0015015 |
| hsa-miR-199a-3p + hsa-miR-199b-3p | — | MIMAT0000232 |
| hsa-miR-191-5p | ASC & BMSC | MIMAT0000440 |
| hsa-miR-100-5p | ASC & BMSC | MIMAT0000098 |
| hsa-miR-127-3p | ASC & BMSC | MIMAT0000446 |
| hsa-miR-1260a | — | MIMAT0005911 |
| hsa-miR-378h | — | MIMAT0018984 |
| hsa-miR-379-5p | — | MIMAT0000733 |
| hsa-miR-376a-3p | — | MIMAT0000729 |
| hsa-let-7i-5p | BMSC | MIMAT0000415 |
| hsa-miR-526a + hsa-miR-518c-5p + | — | MIMAT0002845 |
| hsa-miR-518d-5p | | |
| hsa-miR-212-3p | — | MIMAT0000269 |
| hsa-miR-520c-3p | — | MIMAT0002846 |
| hsa-miR-28-5p | — | MIMAT0000085 |
| hsa-miR-758-3p + hsa-miR-411-3p | — | MIMAT0003879 |
| hsa-miR-29a-3p | — | MIMAT0000086 |
| hsa-miR-1206 | — | MIMAT0005870 |
| hsa-miR-1286 | — | MIMAT0005877 |
| hsa-miR-514a-3p | — | MIMAT0002883 |
| hsa-miR-548ah-5p | — | MIMAT0018972 |
| hsa-miR-184 | — | MIMAT0000454 |
| hsa-miR-543 | — | MIMAT0004954 |
| hsa-miR-626 | — | MIMAT0003295 |
| hsa-miR-339-3p | — | MIMAT0004702 |
| hsa-miR-1234-3p | — | MIMAT0005589 |
| hsa-miR-155-5p | | MIMAT0000646 |
| hsa-miR-888-5p | — | MIMAT0004916 |
| hsa-miR-542-3p | — | MIMAT0003389 |
| hsa-miR-514b-5p | — | MIMAT0015087 |
| hsa-miR-548m | — | MIMAT0005917 |
| hsa-miR-30e-5p | — | MIMAT0000692 |
| hsa-miR-1290 | — | MIMAT0005880 |
| hsa-miR-1255a | | MIMAT0005906 |

The profile of these 39 miRNAs was compared to those published (Baglio et al, 2015) of bone marrow derived mesenchymal stem cell (BMSC) and adipose derived mesenchymal stem cells (ASC). The results demonstrate that the hOMSC secretome miRNA profile is unique. Only 5 and 3 miRNAs are shared between hOMSC secretome miRNAs and the miRNAs detected in BMSC and ASC secretome, respectively. Three out of the 5 miRNAs shared by hOMSC and BMSC secretomes are also shared with ASC secretome. Thus, 34 miRNAs are not contained within the miRNA composition of adult BMSC and ASC secretomes which are considered to be endowed with high therapeutic capacity.

### Example 3. Therapeutic Capacity of hOMSC in Wound Healing

Wound healing in diabetics is delayed due to impaired local and systemic signaling and inappropriate tissue response to wound healing cues. This multifactorial impaired wound healing processes brings about delayed cell migration, reduced new vasculature and connective tissue formation. Stem cells because of their multifactorial secretome have been proposed as cutting-edge tools for the treatment of diabetic wound.

Transgenic mice that lack the leptin receptor (db/db mice), have increased food intake and as a result, become obese and develop type II diabetes, were used as having disease etiology similar to type II diabetes in humans. Diabetic db/db mice exhibit the slowest rate of skin wound closure amongst other known models of diabetes in mice (Michaels J et al. 2007).

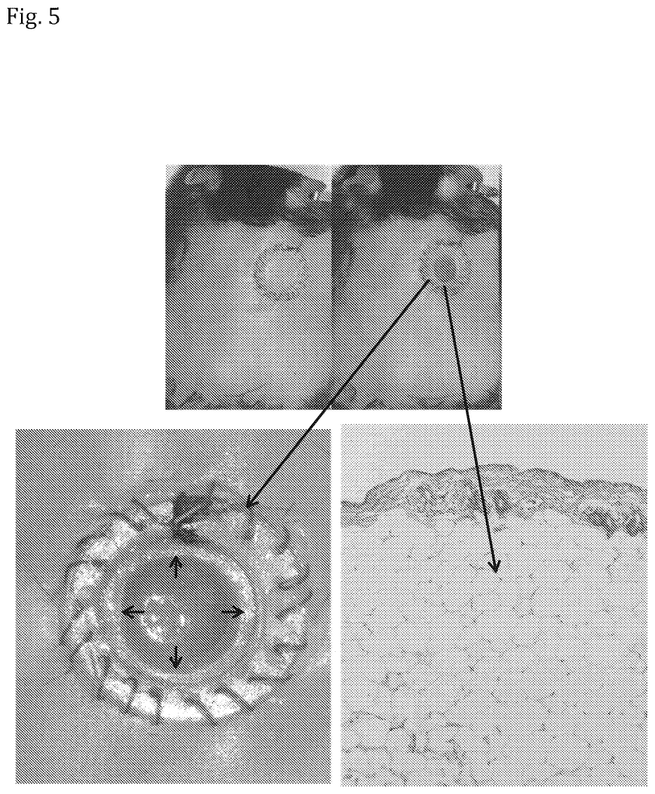

Full thickness dermal wounds 6 mm in diameter were performed on the back of diabetic (blood glucose >300 mg/dcl) db/db mice. A silicon donut-shaped ring having an internal diameter of 8 mm was sutured at the periphery of the wound to prevent wound contraction and served as a standard reference object to calculate the rate of wound closure at the macroscopic level. The animals were divided into 3 groups of 5-10 animal in each: i) a negative control group injected with PBS that served as the vehicle for cell delivery; ii) a hOMSC-treated group; and iii) a hSkin SC treated group. The cells were injected intradermally at 4 equidistant sites, 5×10⁵ cells/site. The animals were photographed every 2-4 days to determine the rate of wound closure. To do this the wound area in each animal at each time point was determined by image analysis on the photographs and the wound area was normalized by determining the area delineated by the inner circumference of the donut-shaped ring as it appeared on the same photograph (FIG. 5).

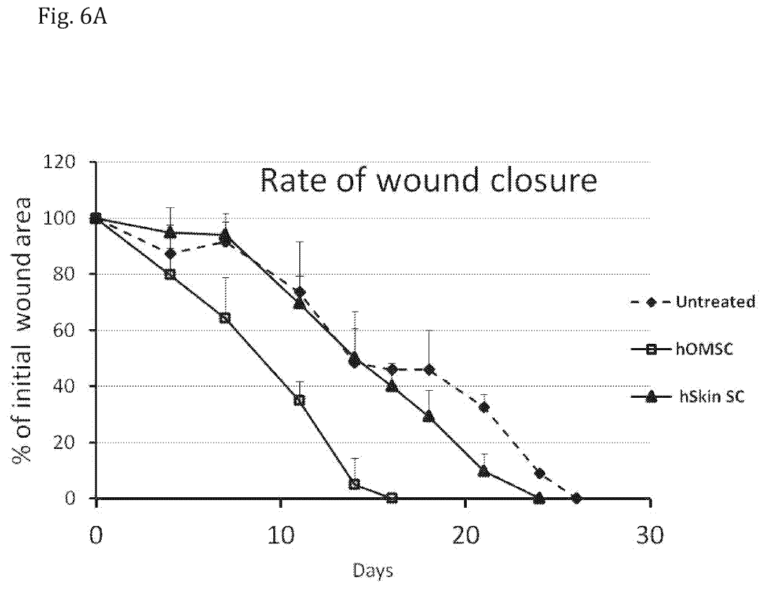

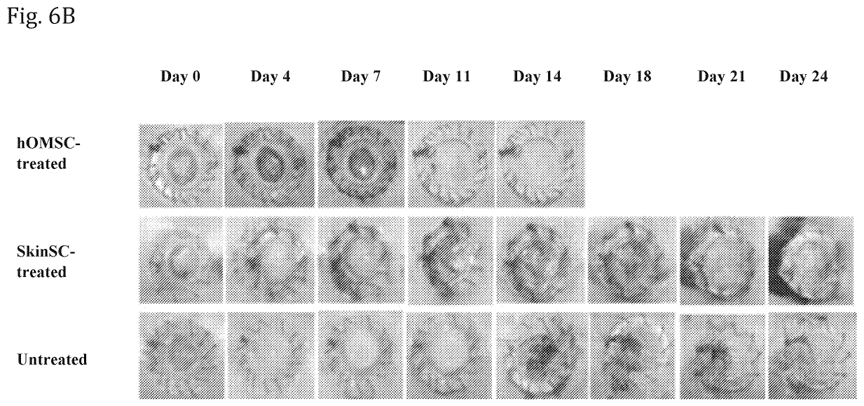

The results demonstrated in FIG. 6A (quantitative results) and 6B (representative qualitative phototgraphs) indicate that the rate of wound healing until wound closure was statistically significant (p<0.05) higher in the hOMSC-treated animals compared to the untreated (PBS vehicle) or the hSkin SC-treated ones. Wound closure in all animals treated with hOMSC occurred 16 days after wounding whereas complete wound closure in all animals of the untreated group took place 26 days after wounding. Furthermore, no significant statistical differences were observed between the untreated group and the hSkin SC treated one.

In a separate study, the natural rate of wound healing was tested also in healthy untreated wild type (WT) animals. For this purpose, the same experimental setting as described above was used. Animals were age-matched with their db/db counterparts.

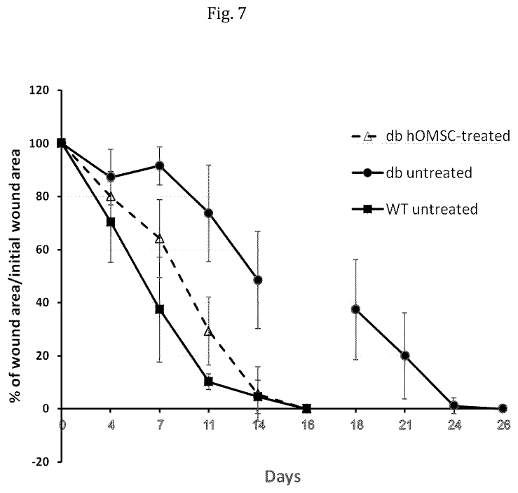

As depicted in FIG. 7, the rate of wound healing in the hOMSC-treated mice was similar to that WT-untreated mice. The average time to complete wound closure in the db/db diabetic mice treated with hOMSC was 14.3±1.4 days, that of the WT-untreated animals was 15.14±1.06 days and that of db/db diabetic untreated mice was 22.75±2.16. These data indicate that hOMSC have the capacity to overcome the deleterious effect of the diabetic status on wound healing and reverse the rate of diabetic wound healing to normal.

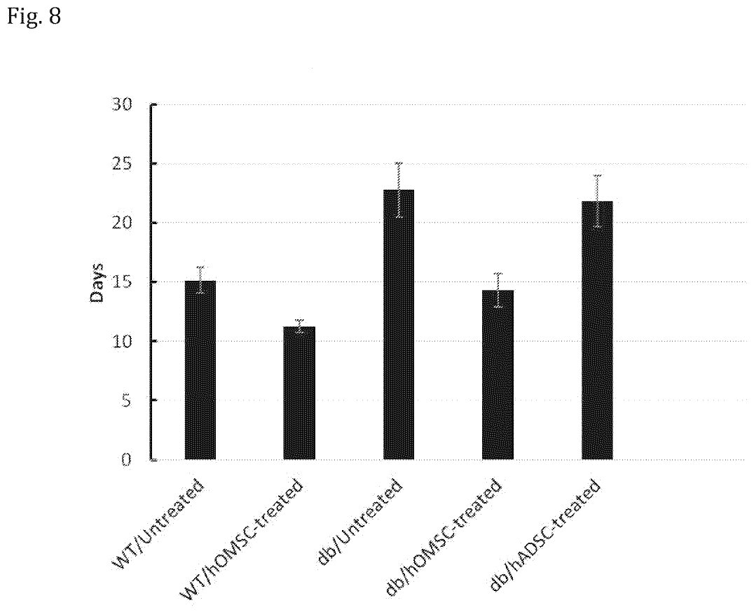

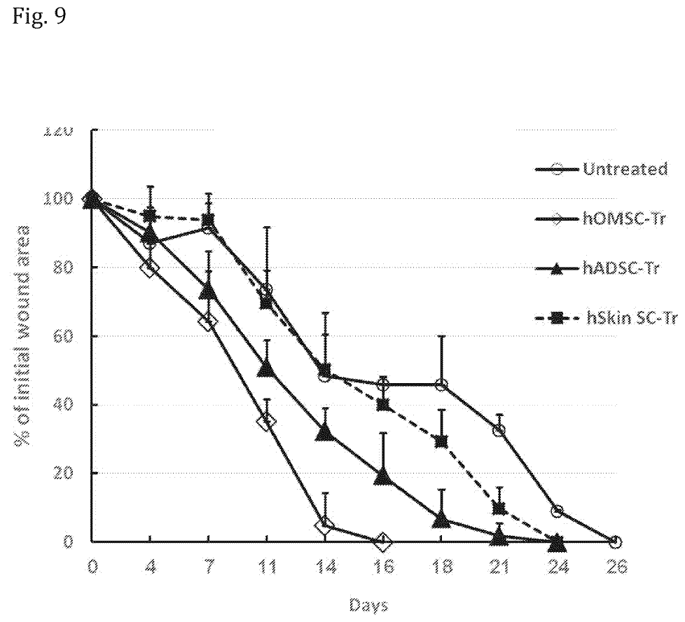

The lack of stimulatory effect of hSkin SC on the of diabetic wound healing was surprising and therefore the capacity of another SC population, namely adipose tissue-derived stem cells (hADSC) was tested in the same experimental setting. The results shown in FIGS. 8 and 9 demonstrate that the rate of wound healing in the hADSC-treated animals was statistical significantly lower than that in the hOMSC-treated animals but higher than that of the untreated- or hSkin SC-treated animals (p<0.05). Nevertheless, complete wound healing in all the animals treated with hADSC occurred 24 days after wounding as for the animals treated with hSkin SC.

It was therefore concluded that Naïve hOMSC are superior to other stem cells in enhancing diabetic wound healing.

### Example 4. The Therapeutic Potential of hOMSC Secretome

To test whether the therapeutic effect of hOMSC can be at least partially attributed to their unique secretome, 1×10⁶ hOMSC were maintained in serum free medium for 24 hours.

Then, the medium was collected and concentrated with a concentration filter having a cutoff of 1,000 Dalton (Amicon). The concentrated medium contains the components of the secretome of naïve hOMSC as analyzed by MS. This hOMSC-derived concentrated conditioned medium was injected in the diabetic wound healing model described in Example 2 and FIG. 5. Each diabetic db/db mice at each site marked by an arrow in FIG. 5 was injected with 50 μl of conditioned medium representing the secretion of 2×10⁵ hOMSC over a period of 24 hours. Thus, a total 200 μl of concentrated conditioned medium was injected into the periphery of the wound of each animal. The administration of the conditioned medium was performed only at the beginning of the experimental period. The animals were followed macroscopically as described above and sacrificed at closure. The wound area including the silicon ring were retrieved and processed for histologic analysis.

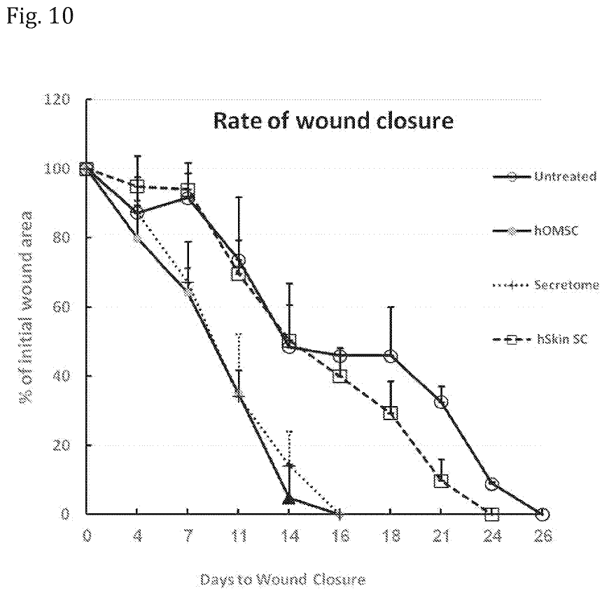

The rate of wound closure is shown in FIG. 10. The results indicate that a one-time administration of hOMSC-secretome confined within the concentrated hOMSC conditioned medium was as effective as hOMSC to enhance diabetic wound healing. Histomorphometric analysis of the number of blood vessels and the amount of collagen in the center of the healed wound revealed: i) no statistical differences between the amount of collagenous connective tissue in the hOMSC-treated animals and that in the hOMSC secretome-treated animals; and ii) increase in the number of blood vessels in the hOMSC secretome-treated animals compared to the untreated or hSkin SC-treated animals and reduction in the number of blood vessels compared to hOMSC-treated animals.

Without wishing to be bound to any theory or mode of action, it is suggested that continued or multiple treatment with secretome will be required compared to treatment with stem cells which continue to secrete substances. Nevertheless, secretome composition is safer and easier to manipulate, characterize, maintain and administer, then cells.

Collectively, the results indicate that the hOMSC-secretome retains the efficiency of hOMSC to enhance the healing of diabetic foot ulcer and therefore they might be used as a self-standing therapeutic tool or as an adjunctive for cell therapy particularly whenever de novo vascularization is required.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### REFERENCES

Antonyak M A, Cerionee R A (2015) Emerging picture of the distinct traits and functions of microvesicles and exosomes. PNAS 112(12), 3589-3590.
Baglio S R, Rooijers K, Koppers-Lalic D. Verweij F J, M P, Zini N, Naaijkens B, Perut F, Niessen H W M, Baldini N and Pegtel D M (2015). Human bone marrow— and adiposemesenchymal stem cells secrete exosomes enriched in distinctive miRNA and tRNA species. 6, 127-147.
Desrochers L M, Antonyak M A, Cerione R A (2016) Extracellular vesicles: Satellites of information transfer in cancer and stem cell biology. Development Cell 37, 301-309.
DiPietro L A (2003). Differential injury responses in oral mucosal and cutaneous wounds. J. Dent. Res. 82, 621-626.
Ganz J, Arie I, Ben-Zur T, Dadon-Nachum M, Pour S, Araidy S, Pitaru S, Offen D (2014). Astrocyte-like cells derived from human oral mucosa stem cells provide neuroprotection in vitro and in vivo. Stem Cells Transl. Med. 3, 375-86.
Ganz J, Arie I, Buch S, Zur T B, Barhum Y, Pour S, Araidy S, Pitaru S, Offen D (2014). Dopaminergic-like neurons derived from oral mucosa stem cells by developmental cues improve symptoms in the hemi-parkinsonian rat model. PLoS One 9(6), e100445.
Gue W., Gao Y., Li N., Shao F., Wang C., Wang P., Yang Z., Li R., and He J. (2017). Exosomes: New players in cancer (Review). Oncology Reports 38: 665-675.
Hu L, Wang J, Zhou X, Xiong Z, Zhao J, Yu R, Huang F, Zhang H, Chen L (2016). Exosomes derived from human adipose mesenchymal stem cells accelerates cutaneous wound healing via optimizing the characteristics of fibroblasts. Scientific Reports 6, 32993.
Kanada M, Bachmann M H, Hardy J W, Frimannson D O, Bronsart L, Wang A, Sylvester M D, Schmidt T L, Kaspar R L, Butte M J, Matin A C, Contag C H (2015). Differential fates of biomolecules delivered to target cells via extracellular vesicles. Proc. Natl. Acad. Sci. USA 112, E1433-E1442.
Konala V B, Mamidi M K, Bhonde R, Das A K, Pochampally R, Pal R (2016). The current landscape of the mesenchymal stromal cell secretome: A new paradigm for cell-free regeneration. Cytotherapy 18(1), 13-24.
Lai, R. C., Arslan, F., Lee, M. M., Sze, N. S. K., Choo, A., Chen, T. S., Salto-Tellez, M., Timmers, L., Lee, C. N., El Oakley, R. M., et al. (2010). Exosome secreted by MSC reduces myocardial ischemia/reperfusion injury. Stem Cell Res. 4, 214-222.
Lopez-Verrilli M A, Caviedes A, Cabrera A, Sandoval S, Wyneken U, Khoury M (2016) Mesenchymal stem cell-derived exosomes from different sources selectively promote neuritic outgrowth. Neuroscience 320, 129-139.
Marynka-Kalmani K, Treves S, Yafee M, Rachima H, Gafni Y, Cohen M A, Pitaru S (2010). The lamina propria of adult human oral mucosa harbors a novel stem cell population. Stem Cells 28(5), 984-95.
Michaels J 5th, Churgin S S, Blechman K M, Greives M R, Aarabi S, Galiano R D, Gurtner G C (2007). db/db mice exhibit severe wound-healing impairments compared with other murine diabetic strains in a silicone-splinted excisional wound model. Wound Repair Regen. 15(5), 665-70
Park C W, Kim K S, Bae S, Son H K, Myung P K, Hong H J, Kim H (2009). Cytokine secretion profiling of human mesenchymal stem cells by antibody array. Int J Stem Cells 2(1), 59-68.
Rodrigues M, Wong V W, Rennert C R, Davis C R, Longaker M T, Gurtner G C (2015). Progenitor cell dysfunction underlie some diabetic complications. The American Journal of Pathology 2015, 85(8).
Skog J, Wurdinger T, van Rijn S, Meijer D H, Gainche L, Sena-Esteves M, Curry W T Jr, Carter B S, Krichevsky A M, Breakefield X O (2008). Glioblastoma microvesicles transport RNA and proteins that promote tumour growth and provide diagnostic biomarkers. Nat. Cell. Biol. 10(12), 1470-6.
Tachida Y, Sakurai H, Okutsu J, Suda K, Sugita R, Yaginuma Y, Ogura Y, Shimada K, Isono F, Kubota K and Kobayashi H. (2015). Proteomic Comparison of the Secreted Factors of Mesenchymal Stem Cells from Bone Marrow, Adipose Tissue and Dental Pulp. Journal of Proteomics and Bioinformatics; 8(12), 266-273.
Tepper O M, Car J, “Robert J. Allen Jr, Chang C C,” Li C D, Tanaka R, Gupta S M, Levine J P, Saa deh P B, Warren S M. (2010). Decreased circulating cell number and failed mechanisms of stromal cell-derived factor-1α mediated bone marrow mobilization impair diabetic tissue repair. Diabetes 59, 1974-1983.
Treves-Manusevitz S, Hoz L, Rachima H, Montoya G, Tzur E, Vardimon A, Narayanan A S, Amar S, Arzate H, Pitaru S (2013). Stem cells of the lamina propria of human oral mucosa and gingiva develop into mineralized tissues in vivo. J. Clin. Periodontol. 40(1), 73-81.
Villarroya-Beltri C, Baixauli F, Gutierrez-Vazquez C, Sanchez-Madrid F, Mittelbrunn M (2014) Sorting it out: regulation of exosome loading. Semin Cancer Biol 28, 3-13.
Voutilainen M H, Arumae U, Airavaara M, Saarma M. (2015). Therapeutic potential of the endoplasmatic reticulum located and secreted CDNF/MANF family of neurotrophic factors in Parkinson's Disease FEBS Letter 589, 3739-3748.
Wu Y., Peng H., Cui M., Whitney N. P., Huang Y., and Zheng C. (2009), CXCL2 increases human neural progenitor cell proliferation through Akt-1/FOXO3a signaling pathway. Journal of Neurochemistry 109, 1157-1167.
Xu R, Greening D W, Zhu H J, Takahashi N, Simpson R J (2016). Extracellular vesicle isolation and characterization: toward clinical application. J, Clin, Invest.; 126(4), 1152-62.
Zander C, Heidbreder M, Kasperek Y, Noll T, Seitz O, Saldamli B, Sudhoff H, Sader R, Kaltschmidt C, Kaltschmidt B (2009). Adult Palatum as a Novel Source of Neural Crest-Related Stem Cells. Stem Cells; 27, 1899-1910.
Zhang Q, Nguyen P, Xu Q, Park W, Lee S, Furuhashi A, Le A D (2017). Neural Progenitor-Like Cells Induced from Human Gingiva-Derived Mesenchymal Stem Cells Regulate Myelination of Schwann Cells in Rat Sciatic Nerve Regeneration. Stem Cells Transl. Med. 6(2), 458-470.

1. A method of treating a disease or disorder comprising administering to a subject in need thereof a cell-free pharmaceutical composition comprising substances secreted from human oral mucosa stem cells (hOMSC-derived secretome), together with at least one carrier, excipient, or diluent, wherein the method involves tissue remodeling, tissue repair or tissue regeneration.

2. The method of claim 1, wherein the cell-free pharmaceutical composition comprises:
(i) The proteins: Stromal cell-derived factor 1 (CXCL12/SDF1), Superoxide dismutase [Cu—Zn] (SOD1), Mesencephalic astrocyte-derived neurotrophic factor (MANF), Cystatin-C(CST3), Galectin-1 (LGALS1), Glia-derived nexin (SERPINE2), Insulin-like growth factor II (IGF2), Latent-transforming growth factor beta-binding protein 1 (LTBP1), Latent-transforming growth factor beta-binding protein 2 (LTBP2), Latent-transforming growth factor beta-binding protein 3 Fragment (LTBP3), Latent-transforming growth factor beta-binding protein 4 (LTBP4), Neuroblast differentiation-associated protein (AHNAK) and Pigment epithelium-derived factor (SERPINF1/PEDF); or
(ii) The proteins: hepatocyte growth factor (HGF), placental growth factor (PIGF), macrophage colony-stimulating factor (MCSF), vascular endothelial growth factor (VEGF), granulocyte colony stimulating factor (GCSF), Macrophage Inflammatory Protein-3 (MIP-3a), growth-regulated oncogene-alpha (GRO-a or CXCL1), Macrophage-Derived/CCL22 Chemokine (MDC or CCL22), growth-regulated oncogene (GRO), IGFBP-2, neurotrophin-4 (NT-4), monocyte chemoattractant protein 2 (MCP-2/CCL8), insulin growth factor-1 (IGF-1), Granulocyte-macrophage colony-stimulating factor (GM-CSF), Interleukin-2 (IL-2) and Brain-Derived neurotrophic factor (BDNF); or
(iii) The proteins: 1 SV, 3 SV, ACTG2, ADAM10, ADAMTSL1, ADM, ANXA4, APOD, CALM2, CD109, CD59, CDH6, CFD, COL15A1, COL1A2, COLEC12, CTHRC1, CTSC, CTSL, CXCL12, DCD, DDAH2, DKK1, DSG1, DSP, DSTN, ECH1, EDIL3, EFEMP1, ELN, FLG, GNB2, GREM2, H3F3B, HBA1, HIST1H2AH, HIST1H2BK, HIST1H4A, HMGN2, HNRNPAB, HSP90AA1, HSPA1A, HSPG2, IGFBP5, JUP, KHSRP, LDHA, MNB2, LTBP4, MAN1A1, MFAP4, MMP1, MMP14, MT2A, NBL1, OMD, PFN1, PI16, PSG5, PSMB6, PTGDS, RARRES2, SLIT3, SPOCKI, SPTBN4, STOM, TMSB10, TMSB4X, TNFAIP6, TNXB, TPI1, TUBA1C, UBC, VIT and WNT5A; or
(iv) the microRNA (miRNA) molecules: hsa-miR-4454+hsa-miR-7975, hsa-miR-23a-3p, hsa-let-7b-5p, hsa-miR-612, hsa-miR-125b-5p, hsa-miR-3144-3p, hsa-miR-199a-3p+hsa-miR-199b-3p, hsa-miR-191-5p, hsa-miR-100-5p, hsa-miR-127-3p, hsa-miR-1260a, hsa-miR-378h, hsa-miR-379-5p, hsa-miR-376a-3p, hsa-let-7i-5p hsa-miR-526a+hsa-miR-518c-5p+hsa-miR-518d-5p, hsa-miR-212-3p, hsa-miR-520c-3p, hsa-miR-28-5p, hsa-miR-758-3p+hsa-miR-411-3p, hsa-miR-29a-3p, hsa-miR-1206, hsa-miR-1286, hsa-miR-514a-3p, hsa-miR-548ah-5p, hsa-miR-184, hsa-miR-543, hsa-miR-626, hsa-miR-339-3p, hsa-miR-1234-3p, hsa-miR-155-5p, hsa-miR-888-5p, hsa-miR-542-3p, hsa-miR-514b-5p, hsa-miR-548m, hsa-miR-30e-5p and hsa-miR-1290;
or combinations thereof.

3. The method of claim 1, wherein the cell-free pharmaceutical composition comprises the substances from (i), (ii), (iii), and (iv).

4. The method of claim 1, wherein the cell-free pharmaceutical composition comprises at least one factor selected from the group consisting of: Stromal cell-derived factor 1 (CXCL12/SDF1), Mesencephalic astrocyte-derived neurotrophic factor (MANF), Superoxide dismutase [Cu—Zn] (SOD1), hepatocyte growth factor (HGF), placental growth factor (PIGF), macrophage colony-stimulating factor (MCSF) and vascular endothelial growth factor (VEGF).

5. The method of claim 1, wherein the cell-free pharmaceutical composition comprises the microRNA molecules: hsa-miR-4454+hsa-miR-7975, hsa-miR-23a-3p, hsa-let-7b-5p, hsa-miR-612, hsa-miR-125b-5p, hsa-miR-3144-3p, hsa-miR-199a-3p+hsa-miR-199b-3p, hsa-miR-191-5p, hsa-miR-100-5p, hsa-miR-127-3p, hsa-miR-1260a, hsa-miR-378h, hsa-miR-379-5p, hsa-miR-376a-3p, hsa-let-7i-5p, hsa-miR-526a+hsa-miR-518c-5p+hsa-miR-518d-5p, hsa-miR-212-3p, hsa-miR-520c-3p, hsa-miR-28-5p, hsa-miR-758-3p+hsa-miR-411-3p, hsa-miR-29a-3p, hsa-miR-1206, hsa-miR-1286, hsa-miR-514a-3p, hsa-miR-548ah-5p, hsa-miR-184, hsa-miR-543, hsa-miR-626, hsa-miR-339-3p, hsa-miR-1234-3p, hsa-miR-155-5p, hsa-miR-888-5p, hsa-miR-542-3p, hsa-miR-514b-5p, hsa-miR-548m, hsa-miR-30e-5p and hsa-miR-1290.

6. The method of claim 1, wherein at least one protein selected from the group consisting of: Stromal cell-derived factor 1 (CXCL12/SDF1, P48061), Superoxide dismutase [Cu—Zn] (SOD1, P00441), Mesencephalic astrocyte-derived neurotrophic factor (MANF, P55145), hepatocyte growth factor (HGF), placental growth factor (PIGF), macrophage colony-stimulating factor (MCSF), and vascular endothelial growth factor (VEGF), is present in the cell-free pharmaceutical composition in a significant higher concentration than in secretome derived from skin or bone marrow stem cells.

7. The method of claim 1, wherein at least one protein selected from the group consisting of: Interleukin-8 (IL-8), Monokine induced by gamma interferon (MIG/CXCL9), Interleukin-6 (IL-6), Fms-related tyrosine kinase 3 ligand (Flt-3 ligand), Leptin, epithelial-derived neutrophil-activating peptide 78 (ENA-78/CXCL5) and monocyte-chemotactic protein 3 (MCP-3/CCL7), is present in the cell-free pharmaceutical composition in a significant lower concentration than in secretome derived from skin or bone marrow stem cells.

8. The method of claim 1, wherein the cell-free composition comprises at least one protein involved in the homeostasis of the nervous system, wherein said protein is selected from the group consisting of: Cystatin-C, Galectin-1, Glia-derived nexin, Insulin-like growth factor II, (IGF2), Latent-transforming growth factor beta-binding protein 1 (LTBP1), Latent-transforming growth factor beta-binding protein 2 (LTBP2); Latent-transforming growth factor beta-binding protein 3 (LTBP3); Latent-transforming growth factor beta-binding protein 4 (LTBP4); Mesencephalic astrocyte-derived neurotrophic factor (MANF), Neuroblast differentiation-associated protein (AHANK), Pigment epithelium-derived factor (PEDF), Stromal cell-derived factor 1 (SDF1), and Superoxide dismutase [Cu—Zn] (SODC).

9. The method of claim 1, wherein the cell-free composition comprises soluble factors, extracellular vesicles (EV), microvesicles and/or exosomes.

10. The method of claim 1, wherein the cell-free pharmaceutical composition comprises soluble factors having a molecular size of 1,000 Daltons or higher.

11. The method of claim 1, comprising at least one process selected from the group consisting of: enhancing wound healing, preventing or reducing scar formation, enhancing scar healing or enhancing cartilage- or bone-formation; enhancing repair or regeneration of the central nervous system or the peripheral nervous system caused by trauma, neurodegenerative disease or vascular diseases of the neural system; and enhancing neo-angiogenesis and neovascularization of an ischemic organ.

12. The method of claim 11, comprising repair or regeneration of organs and tissues that were totally or partially destroyed by at least one iatrogenic injury.

13. The method of claim 12, wherein the at least one iatrogenic injury is caused by mechanical trauma, chemical injury, chemotherapy, radiation or heat.

14. The method of claim 12, wherein the at least one injury is selected from the group consisting of: contusion of the central nervous system, spinal injuries, section of the spinal cord, peripheral nerve crush or section, burns, neuropathy, cardiopathy, bone fractures, tendon and ligament rupture.

15. The method of claim 12 wherein tissue remodeling, tissue repair or tissue regeneration comprises at least one process selected from the group consisting of: enhancing wound healing, preventing or reducing scar formation, enhancing scar healing or enhancing cartilage- or bone-formation; enhancing repair or regeneration of the central nervous system or the peripheral nervous system caused by trauma, neurodegenerative disease or vascular diseases of the neural system; and enhancing neo-angiogenesis and neovascularization of an ischemic organ.

16. The method of claim 15 wherein the ischemic organ is selected from the group consisting of heart, brain, peripheral nerves, and kidney.

17. The method of claim 1 wherein the treatment results in protection or repair of the nervous central nervous system or the peripheral nervous system, or in enhancement of diabetic wound healing.

18. The method of claim 1 wherein the cell-free pharmaceutical composition is administered to a subject in need thereof via a route selected from the group consisting of: topically, subcutaneously, intramuscularly, intraarterial, intraperitoneal, intrathecal, intravenously or directly injected in any tissue at the site in need.

19. The method of claim 1 wherein the cell-free pharmaceutical composition comprises secretome derived from autologous or allogeneic hOMSC.

20. The method of claim 1, wherein the hOMSC-derived secretome is derived from naïve cells.
